(19) United States
(12) Patent Application Publication
(10) Pub. No.: US 2020/0009298 A1
(43) Pub. Date: Jan. 9, 2020

(54) A HYDROGEL COMPOSITE

(71) Applicant: NANYANG TECHNOLOGICAL UNIVERSITY, Singapore (SG)

(72) Inventors: Ratima SUNTORNNOND, Singapore (SG); Jia AN, Singapore (SG); Chee Kai CHUA, Singapore (SG); Edgar Yong Sheng TAN, Singapore (SG); Wai Yee YEONG, Singapore (SG); Jie Kai ER, Singapore (SG)

(21) Appl. No.: 16/490,034

(22) PCT Filed: Mar. 1, 2018
(86) PCT No.: PCT/SG2018/050094

§ 371 Date: Aug. 29, 2019

(30) Foreign Application Priority Data
Mar. 1, 2017 (SG) 10201701620X

(51) Int. Cl. A61L 27/48; A61L 27/52; A61L 27/50

(52) U.S. Cl. CPC A61L 27/48; A61L 27/507; A61L 27/52

(57) ABSTRACT

A hydrogel composite is provided. The hydrogel composite comprises a modified poloxamer having a first charge moiety and a peptide having a second charge moiety, wherein the first charge moiety and the second charge moiety are oppositely charged for ionic interaction between the modified poloxamer and the peptide, and wherein at least one of the modified poloxamer and the peptide comprises a crosslinkable moiety. In particular, the modified poloxamer is a pluronic monocarboxylate activated by dimethylaminopyridine and triethanolamine; the peptide is a gelatin methacrylate. A bioshaping method using the hydrogel composite is provided, as well as a three-dimensional network obtained by the bioshaping method.

### CROSS-REFERENCE TO RELATED APPLICATION

This application claims the benefit of priority of Singapore patent application No. 10201701620X filed on 1 Mar. 2017, the content of which is incorporated herein by reference in its entirety for all purposes.

### TECHNICAL FIELD

Various embodiments relate to a hydrogel composite and a method of shaping the hydrogel composite into a three-dimensional network. The three-dimensional network may be applied in tissue engineering.

### BACKGROUND

Organ transplantation is a medical procedure in which an organ is removed from one body and placed in the body of a recipient, to replace a damaged or missing organ. Although the number of patients in need of an organ transplant is growing exponentially, suitable donors are scarce, resulting in limited number of organs for transplantation. Even where a suitable donor is located, problems such as transplant rejection, which may be due to biological incompatibility and patient autoimmune reaction, may arise.

Tissue engineering is a growing field which seeks to combine cellular, molecular, technological and medical advances to create replacement tissues suitable for implantation. The main challenge in tissue engineering is the ability to create vascular network structures, especially for engineering tissues that are thicker than approximately 200 μm. Vasculature-like structures are usually multilayer and hollow, and have a complex shape with varying diameters throughout the body. It is therefore difficult to imitate this complex 3D structure using materials similar to native vessels such as hydrogels. Insufficient vascularization limits the oxygen and nutrient transfer to cells, which can lead to hypoxia and formation of non-uniform tissue structures.

Bioprinting is a precision technology integrating living materials, motion control, computer-aided design (CAD) software and biomaterials with the aim to provide 3D tissue or organs for implantation, tissue models for drug testing and cell-material interaction study. By using a 3D bioprinter, the researchers in this field hope to print customized organs with patients' own cells without immunological issue. However, printing of biological structures with physiological size and complexity is very challenging due to the inherent limitation of the hydrogel bio-ink materials since they usually have low stiffness and poor printing fidelity. Most of the bio-inks have very low printability and only can create 2.5D shape which is not actually a functional 3D structure (as shown in FIG. 1A). Moreover, a vascular network, as required for tissue engineering, is very complex as illustrated in FIG. 1B and this has been a main obstacle in 3D bioprinting, which may be the reason why direct bioprinting of such a complex 3D structure on a solid platform in a single step has not been realised.

Bioprinting technology may be classified into three main categories: laser-based, jetting-based and micro-extrusion-based. Of these, micro-extrusion is the most versatile as it is relatively inexpensive, easy to operate and compatible with a wide range of materials with printable viscosities ranging up to 6×10⁷ mPa·s. Therefore, micro-extrusion is usually selected for printing thermo-responsive hydrogels to cope with viscosity shift upon temperature change. Thermo-responsive hydrogels have been used for many biomedical applications such as dressings for wound healing and scaffolds for tissue engineering, because they have unique sol-gel transition properties that can be tuned by temperature.

Notwithstanding the above, state of the art techniques are dependent on a liquid platform and unable to directly print hydrogel on a non-liquid platform such as wounds. Moreover, previously reported materials cannot be printed at room temperature or human body temperature, due to poor printability and shape fidelity at these temperatures.

Summarizing the above, current hydrogel bio-ink materials do not have suitable printability and mechanical stability after printing. Users are also unable to print hollow cross-linked structures which mimic vascular networks, hence the printed work may collapse due to weak mechanical properties.

As mentioned above, vascularization is one major obstacle in bioprinting and tissue engineering. In order to create thick tissues or organs that can function like original body parts, the presence of a perfusable vascular system is essential. However, it is challenging to bioprint a hydrogel-based three-dimensional vasculature-like structure in a single step.

Hence, there remains a need for improved hydrogel composites and bioshaping methods to synthesize vasculature-like structures that address or at least alleviate one or more of the above-mentioned problems.

### SUMMARY

In a first aspect, a hydrogel composite is provided. The hydrogel composite comprises a modified poloxamer having a first charge moiety and a peptide having a second charge moiety, wherein the first charge moiety and the second charge moiety are oppositely charged for ionic interaction between the modified poloxamer and the peptide, and wherein at least one of the modified poloxamer and the peptide comprises a crosslinkable moiety.

In a second aspect, a process for making a hydrogel composite is provided. The process comprises a) providing a modified poloxamer having a first charge moiety and a peptide having a second charge moiety, wherein the first charge moiety and the second charge moiety are oppositely charged for ionic interaction between the modified poloxamer and the peptide, and wherein at least one of the modified poloxamer and the peptide comprises a crosslinkable moiety, b) mixing the modified poloxamer with the peptide and allowing the modified poloxamer and the peptide to ionically interact to obtain the hydrogel composite.

In a third aspect, a kit of parts for making a hydrogel composite as described above is provided. The kit comprises a first part of a modified poloxamer having a first charge moiety and a second part of a peptide having a second charge moiety, wherein the first charge moiety and the second charge moiety are oppositely charged for ionic interaction between the modified poloxamer and the peptide, and wherein at least one of the modified poloxamer and the peptide comprises a crosslinkable moiety.

In a fourth aspect, a bioshaping method is provided. The method comprises providing the hydrogel composite as described in the first aspect above, shaping the hydrogel composite to obtain a shaped hydrogel composite, and carrying out a cross-linking reaction on the shaped hydrogel composite to obtain a three-dimensional crosslinked network.

In a fifth aspect, a three-dimensional crosslinked network is provided. The three-dimensional crosslinked network is obtainable using the bioshaping method as described in the third aspect above.

In a sixth aspect, a three-dimensional crosslinked network as described in the fifth aspect above is provided for use in therapy.

In a seventh aspect, a method of treating vascularization insufficiency is provided. The method comprises administering to a mammal an effective amount of a three-dimensional crosslinked network as described in the fifth aspect above.

### BRIEF DESCRIPTION OF THE DRAWINGS

The invention will be better understood with reference to the detailed description when considered in conjunction with the non-limiting examples and the accompanying drawings, in which:

FIG. 1A shows photographs of state of the art hydrogels.

FIG. 1B is a picture of a complex blood vessels network as found in the liver.

FIG. 2A is a photograph showing a comparison between pluronic monocarboxylate:gelatin methacrylate (PluMP:GelMA) according to an embodiment disclosed herein and pluronic 127:gelatin methacrylate (Pluronic:GelMA) composite inks.

FIG. 2B shows a structure fabricated using PluMP:GelMA composite according to an embodiment disclosed herein (left) and Pluronic:GelMA composite (right) where arrows indicate the defects that resulted from phase separation.

FIG. 3 is a graph showing the enzymatic degradation test of Plu-GelMA composite according to an embodiment disclosed herein as a comparison between 7.5% GelMA and 15% GelMA.

FIG. 4 shows a Table comparing the prior art and the hydrogel presented herein. With regard to the printing resolution and the shape stability after printing, the presently disclosed hydrogel composite provides for a higher printing resolution (medium to high resolution when being printed (<150 μm)) and a higher shape stability after printing as compared with other state-of-the-art UV-crosslinkable hydrogels (for example, BioInk™ by RegenHU and GelMA). With regard to the mechanical strength and to the stability at 37° C., the presently disclosed hydrogel composite provides for a higher mechanical strength after crosslinking, and for a higher stability at 37° C., as compared with a state-of-the-art hydrogel, for example a poloxamer, such as pluronic, which only has a low mechanical strength and a low stability at 37° C. With regard to the biological affinity, the presently disclosed hydrogel composite provides for a medium to high biological affinity, which is superior to the biological affinities as presented by the hydrogel of BioInk™ by RegenHU and the poloxamer. These advantages allow the presently disclosed hydrogel composite to be used in applications such as bioprinting of 3D complex bio-structures for implantation, tissue models and vascularization.

FIG. 5A is a reaction scheme for the synthesis of pluronic monocarboxylate.

FIG. 5B is a reaction scheme for the synthesis of gelatin methacrylate (GelMA).

FIG. 6A is a schematic diagram showing structure and functional group of reactants.

FIG. 6B is a schematic drawing of Plu-GelMA synthesis (top) and fabrication process (bottom).

FIG. 7 is a schematic drawing of the physical reaction for the synthesis of the Pluronic-GelMA composite.

FIG. 8A shows NMR results of GelMA, Plu-MP and Plu-GelMA, wherein the arrows point to the functional group that is influenced by GelMA.

FIG. 8B shows ATR-FTIR results of UV crosslinked Plu-GelMA at different mass ratios.

FIG. 8C shows a Plu-GelMA rheological study of viscosity vs shear rate of Plu-GelMA composite at different mass ratios.

FIG. 8D shows water swelling ratio of Plu-GelMA hydrogel composite at different mass ratios (n=3, statistical significance determined by pairwise test wherein *P<0.05).

FIG. 9A shows the tensile modulus of Plu-GelMA composites at different mass ratios.

FIG. 9B shows the tensile strain of Plu-GelMA composites at different mass ratios.

FIG. 9C shows the compressive modulus of Plu-GelMA composites at different mass ratios.

FIG. 9D shows the compressive strain of Plu-GelMA composites at different mass ratios (n=3, statistical significance determined by pairwise t-test wherein *P<0.05).

FIG. 10A shows the standard curve for fluorescein isothiocyanate (FITC)-dextran concentration and curve fitting of the Plu-GelMA hydrogel composites.

FIG. 10B shows the FITC release results of different concentrations of the Plu-GelMA hydrogel composite.

FIG. 11A shows the enzymatic degradation study of Plu-GelMA hydrogel composite at different ratios over 16 days in 0.02 wt % collagenase (all groups n=3, error bars represent s.d.).

FIG. 11B is a SEM image at 500× magnification of Plu-GelMA composite showing the morphology of the hydrogel composite at a mass ratio of 1:2.

FIG. 11C is a SEM image at 500× magnification of Plu-GelMA composite showing the morphology of the hydrogel composite at a mass ratio of 1:1.

FIG. 11D is a SEM image at 500× magnification of Plu-GelMA composite showing the morphology of the hydrogel composite at a mass ratio of 2:1.

FIG. 12A is a process flow diagram of 3D complex structure fabrication.

FIG. 12B is a photograph of a three-dimensional network with a cylindrical structure of 50 layers.

FIG. 12C is a photograph of a multilayer structure inspired by blood vessels.

FIG. 12D is a schematic drawing of the printing process, dark ink represents support material and light ink represents model material.

FIG. 12E is a series of photographs showing a hollow vascular branch structure printing file from stereolithography (STL) file to the actual printed part in water.

FIG. 13A is a series of photographs demonstrating a printability test of Plu-GelMA hydrogel composite, wherein a cylindrical shape at 5 layers and 50 layers is printed (scale bars, 5 mm).

FIG. 13B shows photographs demonstrating the repeatability of printing 3D quadfurcated structures while the zoom-in images from 5× microscope present the size of hollow parts inside the structure.

FIG. 13C shows photographs demonstrating the repeatability of printing square grid shapes with eight replicates while the zoom-in images from a 5× microscope present the grid line and spacing.

FIG. 14A shows a perfusion study of a 3D quadfurcated vasculature-like structure, wherein a 3D complex hollow structure fabrication from STL file to photographs showing the actual part in water (arrows point to the hollow part) is shown.

FIG. 14B is a series of photographs demonstrating an air perfusion test (left arrow indicates air inlet tube while right arrows indicate exiting air bubble).

FIG. 14C is a photograph showing a demonstration of a liquid perfusion test.

FIG. 15A shows L929 in vitro cell viability and cell proliferation test, in particular a PrestoBlue cell proliferation test of Plu-GelMA hydrogel from day 1 to day 7 (n=3, statistical significance determined by pairwise t-test where *P<0.05, ** P<0.05 is for significantly different from the rest of the data set).

FIG. 15B is a SEM image, showing the SEM fixation at 500× magnification of L929 cells on Plu-GelMA 2:1.

FIG. 15C shows the live/dead staining for cell viability test of L929 cells on Plu-GelMA 2:1, the images were observed under microscope at 5× magnification (scale bars, 200 μm).

FIG. 16A shows a Human Umbilical Vein Endothelial Cells (HUVECs) in vitro 191 cell evaluation, in particular the live/dead staining and immunofluorescence of HUVECs on Plu-GelMA 2:1, the images were observed under microscope at 20× magnification for actin and collagen IV at day 7 (scale bars, 50 μm) and at 40× magnification for CD31 and Von Willebrand factor (VWF) at day 10 (scale bars, 20 μm).

FIG. 16B is a SEM image, showing the SEM fixation at 1000× magnification of HUVECs on Plu-GelMA 2:1 (day 1) with false colour to present cells attachment.

FIG. 16C is a SEM image, showing the HUVECs on Plu-GelMA 2:1 on day 7.

FIG. 17 shows the high performance printable pluronic hydrogel.

FIG. 18 shows the high mechanical strength of the GelMA hydrogel.

FIG. 19 is a series of photographs demonstrating a comparison of hydrogel printability.

FIG. 20A shows a blood vessel printing process in horizontal orientation, comprising a CAD file and the actual part being printed from it as a photograph.

FIG. 20B shows a hollow structure printing process of 4 branches (side view) comprising a CAD file and the actual part being printed from it as a photograph.

FIG. 20C shows the 4 branches of the hollow structure printing process (front view) of FIG. 20B, where arrow points towards the hollow shape in the structure.

FIG. 21 shows an FTIR comparison between pluronic and PluMP, the box highlights the different fingerprint which is influenced by the presence or absence of a carboxylic chain.

FIG. 22 shows an FTIR comparison between Plu-GelMA composite and GelMA, the boxes which are highlighted show the different fingerprint which arises due to the influence from PluMP.

FIG. 23 shows a ¹H NMR comparison between PluMP (on the top) and Pluronic-GelMA (on the bottom), wherein the arrows point to the different fingerprint which arises due to influence from GelMA.

FIG. 24 shows the rheological properties of pluronic-GelMA composite compared to 24.5% pluronic and 30% pluronic, wherein on the left there is shown the actual unstacking curve, and on the right there is shown the stacking curve.

FIG. 25 is a graph showing the mechanical properties curve of the Plu-GelMA composite.

FIG. 26A shows a microstructure of Plu-GelMA at magnification 500×.

FIG. 26B shows a microstructure of Plu-GelMA at magnification 1000×.

FIG. 26C shows a microstructure of GelMA at magnification 500×.

FIG. 26D shows a microstructure of GelMA at 1000× magnification.

FIG. 27 shows a 3-(4,5-dimethylthiazol-2-yl)-2,5-diphenyltetrazolium bromide (MTT) test using L929 cells at day 1, 3 and 7 on Plu-GelMA composite, 15% GelMA hydrogel and 24 well cell culture plate as positive control.

FIG. 28 shows an immunofluorescent staining of Human Umbilical Vein Endothelial Cells (HUVEC) by using 4′,6-diamidino-2-phenylindole (DAPI), cluster of differentiation 31 (CD31) and Von Willebrand factor (VWF) with 20× fluorescent microscope (scale bar equals to 50 μm).

FIG. 29A is a SEM image showing the SEM fixation of HUVEC cells onto pluronic-GelMA composite at day 3 of culture at 500× magnification (arrows pointed at the cells on the surface).

FIG. 29B is a SEM image showing the SEM fixation of HUVEC cells onto pluronic-GelMA composite at day 7 of culture at 500× magnification.

FIG. 29C is a SEM image showing the SEM fixation of HUVEC cells onto pluronic-GelMA composite at day 3 of culture at 1000× magnification.

FIG. 29D is a SEM image showing the SEM fixation of HUVEC cells onto pluronic-GelMA composite at day 7 of culture at 1000× magnification.

FIG. 30A is a series of photographs demonstrating the liquid perfusion schematic through hollow bioprinted branches structure.

FIG. 30B is a series of photographs demonstrating an air perfusion schematic through hollow bioprinted branches structure.

FIG. 31A is a diagram illustrating the growth of biomaterials in global market.

FIG. 31B is a graph illustrating the growth of bioprinting compared to other technologies.

FIG. 32 shows a scheme for the G-code generation process for printing a 3D hollow structure.

FIG. 33 shows a standard curve for L929 fibroblast cells with curve fittings.

FIG. 34 shows the H&E image of L929 on the Plu-GelMA 2:1 hydrogel composite at 5× (top) and 20× (bottom), wherein the arrows indicate the cell nucleus.

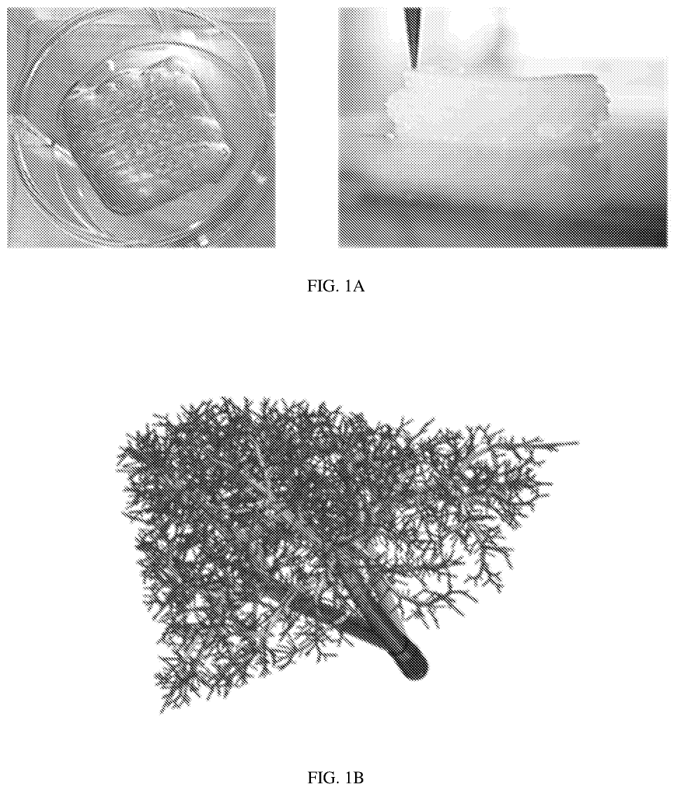

### DETAILED DESCRIPTION

Various embodiments disclosed herein are directed to a hydrogel composite comprising a first component of a modified poloxamer and a second component of a peptide. The modified poloxamer has a high thermo-responsiveness and allows for easy processing and shifting of the hydrogel composite. The peptide, on the other hand, provides protein or cell binding motifs for cell adhesion. By virtue of oppositely charged moieties on the modified poloxamer and the peptide, this provides and/or enables ionic interaction between the modified poloxamer and the peptide. Further, at least one of the modified poloxamer and the peptide comprises a crosslinkable moiety, which may be crosslinked to form a stable, three-dimensional inter-crosslinked network.

The hydrogel-based composite according to embodiments disclosed herein is able to provide improved printability, shape integrity, and biocompatibility for 3D bioshaping of a perfusable complex vasculature-like structure. The hydrogel composite may be used on a non-liquid platform and is shapeable at human body temperature. Moreover, the hydrogel composite is able to support both cell proliferation and cell differentiation. This results in a potentially new vascularization strategy for 3D bioshaping and tissue engineering.

In light of the above, in a first aspect, a hydrogel composite is provided. The composite may comprise a modified poloxamer having a first charge moiety and a peptide having a second charge moiety, wherein the first charge moiety and the second charge moiety are oppositely charged for ionic interaction between the modified poloxamer and the peptide, and wherein at least one of the modified poloxamer and the peptide comprises a crosslinkable moiety.

The term “composite” as used herein refers to a construct including a modified poloxamer having a first charge moiety as a first component and a peptide having a second charge moiety as a second component, wherein the first charge moiety and the second charge moiety are oppositely charged for ionic interaction between the modified poloxamer and the peptide, and wherein at least one of the modified poloxamer and the peptide comprises a crosslinkable moiety. The components in the composite may be physically mixed together to form the composite. By the term “physically mixed”, it is meant that both components are merely dispersed in one another, and do not chemically react to form a new material. Specifically, the modified poloxamer and the peptide are not covalently bonded to each other.

The hydrogel composite comprises a modified poloxamer having a first charge moiety. The term “poloxamer” as used herein refers to a block copolymer made of a chain of polyoxypropylene (the term “poly(propylene oxide)” may be used interchangeably herein) flanked by two chains of polyoxyethylene (the term “poly(ethylene oxide)” may be used interchangeably herein). Poloxamers may be sold under trade names including Pluronic® (BASF), Kolliphor® (BASF), Lutrol® (BASF), and Synperonic® (Croda International). Unless a particular poloxamer species is specified, references to “poloxamer” may generically refer to multiple poloxamer species.

The poloxamer is typically thermo-responsive, meaning that it provides for temperature dependent self-assembling and thermo-gelling behavior. Concentrated aqueous solutions of poloxamers may be liquid at low temperature and may form a gel at higher temperature in a reversible process. The transitions that occur in these systems depend on the polymer composition (molecular weight and hydrophilic/hydrophobic molar ratio). At low temperatures and concentrations (below the critical micelle temperature and critical micelle concentration) individual block copolymers (unimers) may be present in solution. Above these values, aggregation of individual unimers may occur in a process called micellization. This aggregation may be driven by the dehydration of the hydrophobic polyoxypropylene (or poly(propylene oxide), “PPO”) block that becomes progressively less soluble as the polymer concentration or temperature increases. The aggregation of several unimers may occur to minimize the interactions of the PPO blocks with the solvent. Thus, the core of the aggregates may be made from the insoluble blocks poly(propylene oxide) while the soluble portion (poly(ethylene oxide)) forms the shell of the micelles.

Due to these properties, poloxamers, for example pluronic F127 (poloxamer 407), may be used as thermo-responsive hydrogels, wherein the poloxamers have been used as mould, track patterning and sacrificial materials for bioprinting and tissue engineering. Poloxamers may be moved and shifted easily, which is due to the above mentioned inherent ability of micellar-packing gelation. Moreover, the range of their sol-gel transition temperature is broad (for example, 10-40° C. for pluronic F127), meaning that the viscosity of poloxamers may be stable at both room temperature and human body temperature. However, the poor mechanical strength, coupled with their propensity to dissolve in aqueous environments, renders unmodified poloxamers unsuitable for long-term structural support within a tissue scaffold. While poloxamers can be modified with photo-crosslinkable acrylate groups to stabilize the hydrogel, they still lack protein or cell binding motifs, resulting in poor cell adhesion. In the present disclosure, these deficiencies have been addressed or at least alleviated by modifying the poloxamer, which allows for an improved hydrogel composite, providing the advantages of the poloxamer described above while obliterating the shortcomings.

The term “modified poloxamer” refers to a poloxamer which has been modified to contain at least one charged moiety or functional group and this charge is a net charge. This charge may be a negative charge or a positive charge, meaning that the charged functional group may respectively be an anionic functional group or a cationic functional group. The charge may be on a functional group, which is covalently linked to the poloxamer. The charge may arise from a cationic functional group, for example an ammonium ion group. Alternatively, the charge may arise from an anionic functional group, for example from a deprotonated acid, for example a carboxylic acid, a hydroxamic acid, a sulfuric acid or a phosphoric acid. The functionality providing the cationic or anionic charge may be covalently bonded to the poloxamer. The net charge may be present at a particular pH value range or at a pH value, for example at a pH value range of about 5 to 9, or of about 5 to 8, or of about 5 to 7, or of about 6 to 9, or of about 7 to 9, or of about 6 to 8, or at about 7. Preferably, the net charge may be present at physiological pH, which is about pH 7.

In various embodiments, the charge of the modified poloxamer, which is the first charge moiety, may be an anion. Said differently, the first charge moiety may therefore be negatively charged.

In various embodiments, the first charge moiety may be located at a terminal position of the modified poloxamer. The first charge moiety may be connected to the poloxamer with a linker. The modified poloxamer may then have a general structure of formula (I):

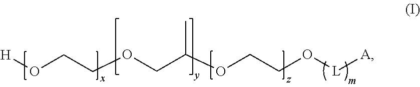

wherein x, y and z are independently integers in the range of about 2 to about 300, m is 0 or 1, L is a linker and A is the first charge moiety.

As mentioned herein and shown in the compound of formula (I), the poloxamer comprises a poly(ethylene glycol) (PEG) and a poly(propylene glycol) (PPG) block. The poly(ethylene glycol) (PEG) and poly(propylene glycol) (PPG) are biocompatible and clearance from the body is possible for blocks of lower molecular weight, for example about 10,000 g/mol. Advantageously, PEG provides hydrophilic blocks that may absorb and retain large quantities of water, while PPG is highly thermosensitive and provides a balanced hydrophobicity and hydrophilicity at different temperatures, facilitating formation of a thermosensitive hydrogel.

In the modified poloxamer, x and z may be identical integers. Hence, the two poly(ethylene glycol) blocks may have the same size.

In various embodiments, x and z are integers in the range of about 40 to 100, or in the range of about 50 to 100, or in the range of about 60 to 100, or in the range of about 50 to 90, or in the range of about 50 to 80, or in the range of about 50 to 70, or in the range of about 60 to 70. In one example, the integers x and z are about 65.

In various embodiments, y may be an integer in the range of about 50 to 150, or in the range of about 50 to 130, or in the range of about 50 to 110, or in the range of about 70 to 150, or in the range of about 90 to 150, or in the range of about 90 to 130, or in the range of about 90 to 110. In one example, the integer y is about 100.

In examples wherein x and z are about 65 and y is about 100, the poloxamer utilized is a poloxamer of the trade name “Pluronic F127”.

A is the first charge moiety, which may be negatively charged. Hence, A may be a deprotonated carboxylic acid, which may also be called a carboxylate (—COO⁻), a deprotonated hydroxamic acid (—C(O)NHO⁻), a phosphate (—OPO₃²⁻), or a sulfate (—OSO₃⁻). In one example, the first charge moiety is a carboxylate.

The first charge moiety A may be covalently attached to the poloxamer with a linker L. In these embodiments, m may be 1. The linker L may be an optionally substituted —C₁₋₂₀alkyl-, optionally substituted —C₂₋₂₀alkylene-, optionally substituted —C₆₋₁₂aryl-, optionally substituted —C₆₋₁₂aryl-C₁₋₂₀alkyl-, which may be optionally substituted or interrupted with an amine, ether, ester, carbonyl, and any combination thereof. The linker may be of a small size. In some embodiments, it may have less than 10 carbon atoms, or less than 5 carbon atoms. Hence, it may be an optionally substituted —C₁₋₉alkyl-, or an optionally substituted —C₁₋₄alkyl-. It may further have less than 2 nitrogen atoms, or no nitrogen atoms. It may have less than 5 oxygen atoms. In some embodiments, the linker L may be —C(O)(CH₂)ₙ—, wherein the carbonyl moiety is covalently bonded to the poloxamer and the alkyl moiety is covalently bonded to A. The linker L may connect the poloxamer with the first charge moiety. In alternative embodiments, the first charge moiety may be covalently connected to the poloxamer directly, without the presence of a linker L. In these embodiments, m may be 0.

In embodiments wherein the anionic charge is a carboxylate, m is 1 and the linker L is —C(O)(CH₂)ₙ—, A in combination with the linker L may be

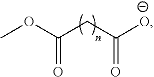

wherein n may be an integer in the range of about 1 to 10, preferably 2.

In various embodiments, the modified poloxamer may have a number average molecular weight in the range of about 5 to about 20 kDa, or of about 5 to about 15 kDa, or of about 10 to about 20 kDa, or of about 10 to about 15 kDa, preferably about 12.5 kDa.

In addition to the modified poloxamer, the hydrogel composite comprises a peptide having a second charge moiety as a second component. The term “peptide” as used herein refers to a molecule with a peptide backbone. Thus, the peptide may refer to a polymer of amino acids linked together by peptide bonds. Depending on the nature of the amino acid, the side chain of the amino acid may optionally be modified. Peptides generally comprise protein or cell binding motifs.

A peptide having a charged moiety and a crosslinkable moiety, as for example gelatin methacrylate (GelMA), has ultraviolet (UV) curable properties, and may be used for vascularized tissue engineering and other biomedical applications. The printability of pure GelMA, however, is generally low and it is difficult to directly print it into a complex 3D structure. In the present disclosure, these deficiencies have been addressed or at least alleviated by combining the peptide having a charged moiety, with a modified poloxamer described above.

Without intending to be limited by theory, it is believed that the ionic interaction of the peptide with the modified poloxamer is generally due to the presence of the charge in the peptide that can electrostatically interact with the opposite charge of the modified poloxamer.

The ionic interaction between the modified poloxamer and the peptide is advantageous as compared to formation of a covalent bond between the two components such as in the case of a copolymer, since the ratio of modified poloxamer component and the peptide that may be used to form the hydrogel composite is independent of stoichiometry, which would otherwise be required for covalent bond-formation.

Moreover, the hydrogel composite is able to stay in a single phase due to the physical ionic bond (ionic interaction) between the charged functional group of the poloxamer and the charge, which is opposite to the charge of the poloxamer, on the peptide, thereby immobilizing the poloxamer component and avoiding having to extensively wash the obtained three-dimensional network, which may affect cell viability during cell culture.

At least one of the modified poloxamer and the peptide comprises a crosslinkable moiety. A plurality of crosslinkable moieties may be present on the modified poloxamer, the peptide or both. The term “crosslinkable” as used herein refers to the property or characteristic of a material to undergo crosslinking upon exposure to crosslinking conditions. This property or characteristic may be the presence of a crosslinkable moiety in one of the two components (modified poloxamer or peptide) in the hydrogel composite. In the present context, the crosslinkable moiety may be an acrylate-based moiety, which may be pendant on the peptide. Accordingly, crosslinking may be effected upon exposure of the hydrogel composite to electromagnetic radiation. The crosslinkable moiety may be covalently attached to one of the two components.

Advantageously, by utilizing both a modified poloxamer having a first charge moiety and a peptide having a second charge moiety, wherein the first charge moiety and the second charge moiety are oppositely charged for ionic interaction between the modified poloxamer and the peptide, and wherein at least one of the modified poloxamer and the peptide comprises a crosslinkable moiety, the mixture of the two components provides for a three-fold gelation or solidification mechanism upon formation of the hydrogel composite, during which covalent and non-covalent associations are being established.

A first gelation mechanism is the ionic interaction between the opposite charges of the two components, which is a physical gelation mechanism. This interaction may be responsible for a “semi-permanent” state of the hydrogel composite. The “semi-permanent” state may be a state, in which the hydrogel composite may still be mouldable, but would not, at a given temperature, change its shape without any exterior influence. The ionic interaction arising from the mixture of these two components may increase the stability between the two components, which may in turn enhance ability of the hydrogel composite to be shaped at a wide range of temperatures.

A second gelation (or solidifying) mechanism is provided in form of a crosslinkable moiety, by which the hydrogel composite may undergo crosslinking, thereby providing a three-dimensional network in a substantially “permanent” state. The “permanent” state may be a state, in which the three-dimensional network may retain its three-dimensional shape after crosslinking without changing its shape under moderate exterior influence, such as the flow of body liquids.

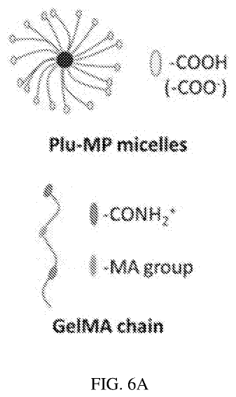

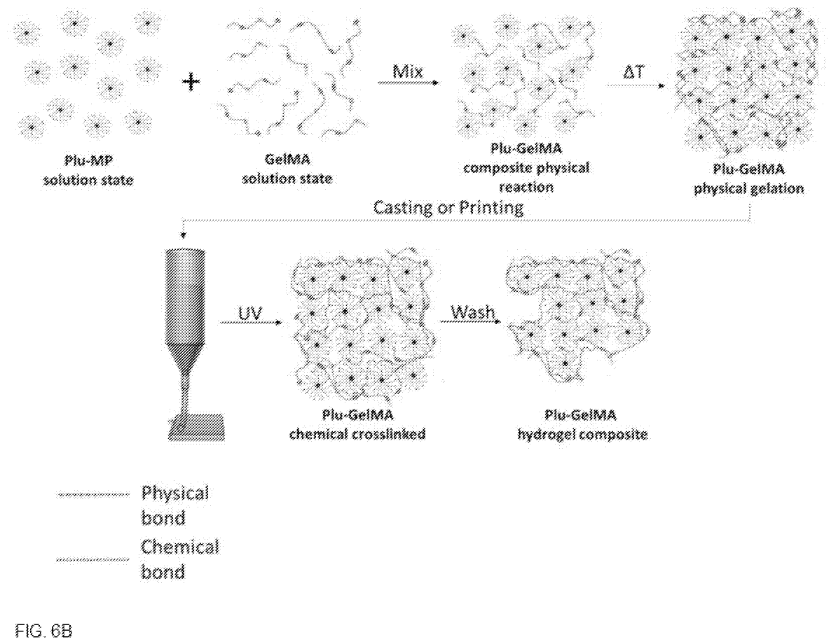

The crosslinked network of either components may extend over and between the other components, thereby impregnating the other component, which is exemplary shown in FIG. 6, thereby providing for a greater degree of an inter-crosslinked network, in addition to the ionic interaction between the two components. The first two gelation or solidifying mechanisms described above arise from the combination of the modified poloxamer and the peptide.

The third gelation mechanism is provided in form of the thermo-responsiveness of the poloxamer, which, as discussed above, allows for easy handling and shaping of the hydrogel composite. By combining all of the three gelation mechanisms in one hydrogel composite, the present disclosure combines the advantages of physically gelled hydrogels, which are expressed in a gentle flow under minor stress, which, in turn, provides good printability, while allowing for the advantages of chemically gelled macromers, being a higher strength within a network. The three-fold gelation mechanism therefore allows for better control and adjustment of key characteristics such as printability, mechanical strength, swelling and flow properties.

In various embodiments, the at least one opposite charge of the peptide is located on the side chain of a charged amino acid. Advantageously, by providing for the ionic interaction with the modified poloxamer on the side chain of the charged amino acid, rather than on the terminal moieties of the peptide, there are more charges provided which allow for the ionic interaction, thereby improving stability of the hydrogel composite.

In embodiments, wherein the first charge moiety is a negative charge, the second charge moiety would then be a positive charge. In some embodiments, the positive charge may be derived from an amino acid, which may then be called a charged amino acid. In embodiments, wherein the second charge moiety may be a positively charged moiety, the charged amino acid may be a cationic amino acid. The term “cationic amino acid” as used herein refers to those amino acids, where the side chains contain a cationic charge. This cationic amino acid is, for example, arginine, lysine and histidine. Thus, the cationic charge may be located on the side chain of arginine, lysine and histidine. The cationic functional group is a functional group that has a net positive charge. The presence of this charge is generally dictated by the pH value of the environment in which the functional group is found. The functional group is represented as charged functional group generally at a physiological pH value (about pH 7). The cationic functional group may be selected from the group consisting of an amidinium ion (—C(NRH⁺)NR₂), an imidazolium ion

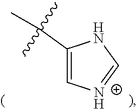 [SMILES: *c1c[nH+]c[nH]1]

an ammonium ion (—NR₃⁺) and any combination thereof. In these embodiments, R may be individually a proton or a C₁₋₁₀alkyl group. In one example, the second charge moiety is selected from an amidinium ion (—C(NRH⁺)NR₂) and the cationic amino acid is arginine.

In various embodiments, the peptide may be a thermo-gelling peptide, for example gelatin. The term “thermo-gelling” as used herein refers to the property or characteristic of a liquid or solution to turn into a non-liquid such as a gel in response to temperature changes. In embodiments, wherein the peptide is a thermo-gelling peptide, the peptide may advantageously provide for a fourth gelling mechanism, thereby providing yet another dimension for controlling the properties and characteristics of the hydrogel composite.

The peptide may be a naturally occurring peptide or a synthetic peptide. In various embodiments, the peptide may be a naturally occurring peptide. The peptide may be selected from the group consisting of sericine, fibroin, elastin, collagen, gelatin and a combination thereof.

In various embodiments, the peptide may be a peptide wherein more than 50% of the amino acids comprised in the peptide are selected from a combination of the group consisting of glycine, proline, hydroxyproline, glutamic acid, alanine, arginine and aspartic acid. In one example, the peptide is gelatin.

As mentioned above, the peptide and/or the modified poloxamer may comprise a crosslinkable moiety. In various embodiments, the crosslinkable moiety may undergo crosslinking through a radical polymerization reaction. In various embodiments, the radical polymerization reaction may be a photo-initiated radical polymerization reaction. The crosslinkable moiety in these embodiments may otherwise be termed as a photo-crosslinkable moiety. In specific embodiments, the photo-crosslinkable moiety may be an acrylate-based moiety. In one example, the acrylate-based moiety is a methacrylate-based moiety.

The crosslinkable moiety may be bonded to an amine functionality of the peptide. This binding may be in the form of a covalent amide bond. The covalent amide bond may be on an electron-rich nitrogen atom of the amino acid side chain. For example, the covalent amide bond may be formed with the amine functionality of a lysine side chain. In this example, the amide bond formation may be carried out under basic conditions in order to provide the lysine side chain in the deprotonated form.

The cationic moiety may be located on the side chain of a cationic amino acid. Cationic amino acids may be selected from those amino acids, where the side chains contain a cationic moiety. As discussed above, those cationic amino acids may be, for example, arginine, lysine and histidine. Thus, the cationic moiety may be located on the side chain of arginine, lysine and histidine. In one example, the cationic moiety may be located on the side chain of arginine. In this example, the cationic moiety may be a terminal amidine moiety, denoted as —C(NH₂⁺)NH₂.

The hydrogel composite may additionally comprise an initiator. The initiator may be a photoinitiator, preferably a radical photoinitiator. A radical photonitiator may be a compound that creates reactive species (free radicals, cations or anions) when exposed to electromagnetic radiation, which may be a radiation in a wavelength of about 10 to about 1000 nm. The photoinitiator may be preferably selected from any radical photoinitiator which creates reactive species under UV light (about 10 to 400 nm). Thus, the photoinitiator may be a photoinitiator to be used in UV curing. In various embodiments, the photoinitiator may be a photoinitiator selected from “irgacure” or “darocur” photoinitiators. In one example, the photoinitiator is an irgacure photoinitiaor, in particular irgacure 2959.

At least one, or both of the modified poloxamer or the peptide, having a second charge moiety, wherein the first charge moiety and the second charge moiety are oppositely charged for ionic interaction between the modified poloxamer and the peptide may be biocompatible. Furthermore, the hydrogel composite resulting from the mixing of the two components may be biocompatible. Additionally, the three-dimensional network obtained after bioshaping of the hydrogel composite may also be biocompatible. As used herein, the term “biocompatible” refers to a component, which does not cause severe toxicity, severe adverse biological reaction, or lethality in an animal when administered at reasonable doses and rates.

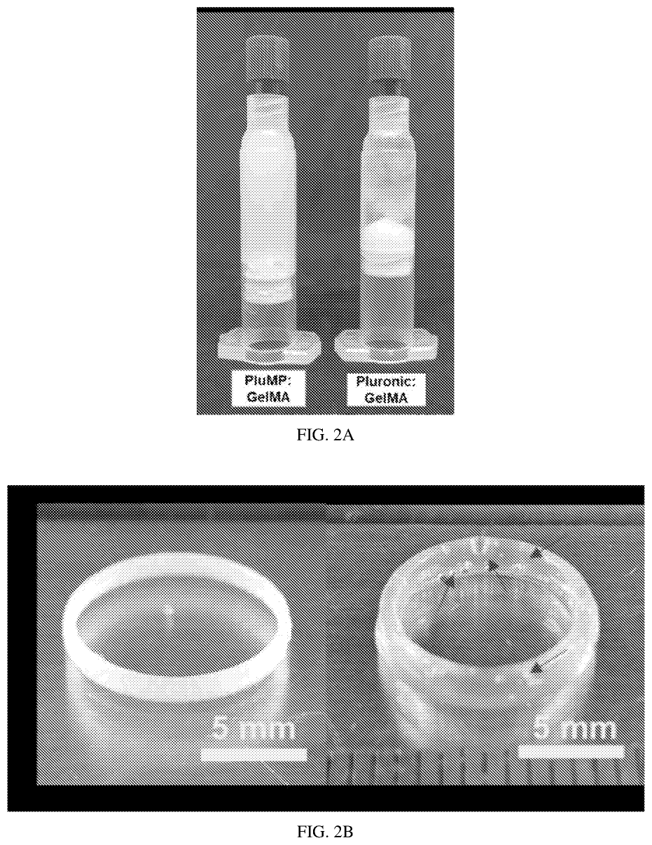

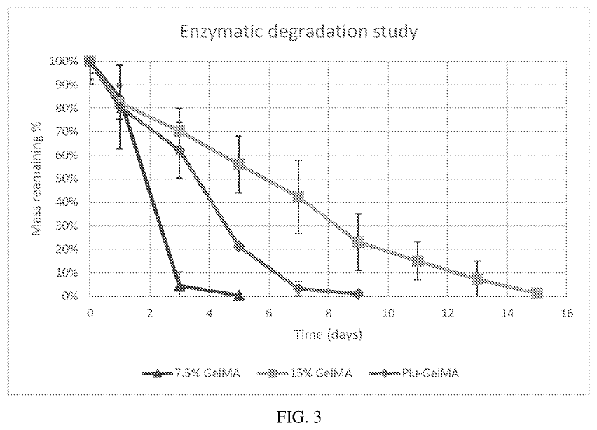

At least one, or both of the charged poloxamer or the peptide, having a second charge moiety, wherein the first charge moiety and the second charge moiety are oppositely charged for ionic interaction between the modified poloxamer and the peptide, may be biodegradable. Furthermore, the hydrogel composite resulting from the mixing of the two components may be biodegradable. Additionally, the three-dimensional network obtained after bioshaping of the hydrogel composite may also be biodegradable. As used herein, the term “biodegradable” refers to components that are capable of being degraded or absorbed when exposed to bodily fluids such as blood, and components thereof such as enzymes or oxidative species, and that can be gradually absorbed and/or eliminated by the body. The biodegradable properties of the hydrogel composite may be measured in an enzymatic degradation study (FIG. 3).

As mentioned above, one advantage of having an ionic interaction between the two components is that the ratio between the components does not have to follow stoichiometric requirements. Hence, in order for the two components to be “connected”, they only require the ionic interaction, which is independent of stoichiometric ratios generally required by covalent bond formation. Thus, the mass ratio of the modified poloxamer to the peptide, wherein the first charge moiety and the second charge moiety are oppositely charged, may be from about 0.5:5 to about 5:0.5, or from about 0.5:4 to about 4:0.5, or from about 0.5:3 to about 3:0.5, or from about 0.5:2 to about 2:0.5, or from about 1:5 to about 5:1, or from about 1:4 to about 4:1, or from about 1:3 to about 3:1, or from about 1:2 to about 3:1, preferably from about 1:1 to about 3:1, more preferably at about 2:1.

Advantageously, at a mass ratio of the components of about 1.8:1 to 2:1, meaning that about double the amount of modified poloxamer is used than the peptide (by weight), the swelling ratio and the porosity of the ensuing three-dimensional network is very high compared to other mass ratios, i.e. this mass ratio results in larger pores by volume in the network. This may result in a higher cell number, which was measured in a cell evaluation and proliferation study.

It is postulated that the larger pores lead to more surface area and better medium transportation through the entire three-dimensional network. As shown in the Examples section, the cells were able to attach onto the hydrogel composites and able to pack together when the cell number increased. This demonstrates that the hydrogel composite with a 2:1 ratio as described above provides a good platform for cell attachment and proliferation. Moreover, at this mass ratio, the swelling ratio may be higher compared to a ratio which has a higher content of the peptide. This, in turn, may result in higher water absorption. Furthermore, at this ratio, the hydrogel composite in its non-crosslinked state is stable at a wide range of temperature similar to a poloxamer alone, thus shaping at cell favourable environment, such as human body temperature, is possible.

Due to the ionic interaction, for example, the hydrogel composite may be in a single phase, meaning that there is no phase separation between the two components of the hydrogel composite. This is demonstrated, for example, in FIG. 2A. FIG. 2A shows a comparative example, wherein the poloxamer of the hydrogel composite in one example is carboxylated, and therefore charged (left), while the poloxamer of the hydrogel composite in the other example is not carboxylated, and therefore not charged (right). It can be seen that the sample with the carboxylated poloxamer is in a single phase, while the sample with the non-carboxylated poloxamer shows a phase separation. The phase separation may result in defects of the formed three-dimensional network arising from such a hydrogel composite, as shown in FIG. 2B. Hence, according to various embodiments, there may be an ionic interaction between the charge of the poloxamer and the opposite charge of the peptide. Advantageously, the charge on the poloxamer may result in having a single phase.

In a second aspect, there is provided a process for making a hydrogel composite. The process comprises
(a) providing a modified poloxamer having a first charge moiety and a peptide having a second charge moiety, wherein the first charge moiety and the second charge moiety are oppositely charged for ionic interaction between the modified poloxamer and the peptide, and wherein at least one of the modified poloxamer and the peptide comprises a crosslinkable moiety,
(b) mixing the modified poloxamer with the peptide and allowing the modified poloxamer and the peptide to ionically interact to obtain the hydrogel composite.

The modified poloxamer may be provided in a 5 to 50 w/v % solution, or in a 5 to 40 w/v % solution, or in a 5 to 30 w/v % solution, or in a 10 to 50 w/v % solution, or in a 20 to 50 w/v % solution, or in a 20 to 40 w/v % solution, or in a 25 to 40 w/v % solution, or in a 25 to 35 w/v % solution. In one example, the modified poloxamer is provided in a 30 w/v % solution.

The peptide may be provided in a 5 to 50 w/v % solution, or in a 5 to 40 w/v % solution, or in a 5 to 30 w/v % solution, or in a 5 to 20 w/v % solution, or in a 5 to 15 w/v % solution, or in a 10 to 50 w/v % solution, or in a 15 to 50 w/v % solution, or in a 15 to 40 w/v % solution, or in a 15 to 30 w/v % solution, or in a 15 to 20 w/v % solution, or preferably in a 10 to 20 w/v % solution. In one example, the peptide is provided in a 15 w/v % solution.

The solvent for the solution of the modified poloxamer and/or the peptide may be a buffer solution. In one example, a phosphate-buffered saline (PBS) solution as a buffer was used. Advantageously, by being in a buffer solution, the pH of the solution may be kept at a value which allows for the modified poloxamer to be present in the charged form and/or for the charge of the peptide to be present, for example through protonation or deprotonation, thus allowing for the ionic interaction to occur once the two components are to be mixed with each other. Further advantageously, this buffer solution may neutralize any acidic components resulting from the reaction in which the crosslinking moiety is attached, such as an acrylic acid in case an acrylic anhydride is used during attachment of the crosslinking moiety. By being in a buffer solution, it is therefore possible to raise the pH value to an acceptable value in order to provide a more cell favourable environment.

In one example, the modified poloxamer is provided in a 30 w/v % solution, while the peptide is provided in a 15 w/v % solution. Hence, in this example, the mass ratio between the two components would be described as 2:1.

The process may further comprise adding an initiator prior to or while mixing the modified poloxamer with the peptide. The initiator may preferably be a photoinitiator, which may be added to the mixture together with the solution comprising the modified poloxamer or the solution comprising the peptide. Preferably, the initiator may be provided in the solution comprising the peptide. The initiator may be provided in a 0.05 to 1 w/v % solution, or in a 0.1 to 1 w/v % solution, or in a 0.15 to 1 w/v % solution, or in a 0.2 to 1 w/v % solution, or in a 0.05 to 0.5 w/v % solution, or in a 0.05 to 0.4 w/v % solution, or preferably in a 0.1 to 0.5 w/v % solution. As mentioned before, the solvent may be a buffer solution.

As mentioned herein, the modified poloxamer may be in the form of micelles, and may be provided in the mixture in the form of micelles. Advantageously, the modified poloxamer is able to exhibit thermo-responsiveness. This may enable the hydrogel composite to be shaped at a temperature suitable for biomedical applications, while the excess material may be removed by washing with cold water.

In various embodiments, the peptide is provided in the form of uncoiled chains of a peptide backbone. The term “uncoiled chains” may refer to the peptide being in a “linear” arrangement, as supposed to be intertwined in a helical form, as observed for other peptides such as DNA segments. Advantageously, by being in its uncoiled form, the peptide may be in a better position for the formation of ionic interaction with the modified poloxamer.

The mixing may be carried out using a three-way stopcock. Advantageously, by using a three-way stopcock, the energy was added into the mixture similarly to the use of a homogenizer. The energy that was added into the system might lead to the formation of a stable foam structure inside the hydrogel.

The mixing may result in a physical reaction between the two components. The term “physical reaction” is used herein as the opposite to a “chemical reaction”, wherein covalent bonds may be formed or broken. Thus, the physical reaction may refer to electrostatic effects, which would increase the affinity of the charged functional group on the peptide to engage with the modified poloxamer, thereby forming an ionic interaction.

After mixing, the ensuing mixture may be kept for about 1 to about 100 hours, or for about 1 to about 80 hours, or for about 1 to about 60 hours, or for about 1 to about 40 hours, or for about 1 to about 20 hours, or for about 3 to about 100 hours, or for about 5 to about 100 hours, or for about 10 to about 100 hours, or for about 3 to about 70 hours, or for about 3 to about 30 hours, or for about 5 to about 15 hours.

The temperature at which the mixture is kept may be from about 10° C. to about 50° C., or from about 15° C. to about 50° C., or from about 20° C. to about 50° C., or from about 10° C. to about 40° C., or from about 10° C. to about 30° C., or from about 15° C. to about 25° C., or about room temperature.

During this time and/or at this temperature, the modified poloxamer may be allowed to ionically interact with the peptide and the solution's viscosity may increase. This step may refer to physical gelation. Hence, physical gelation may refer to the formation of the ionic interaction, which ultimately provides more stability, in order to form the crosslinkable hydrogel composite.

In a third aspect, there is provided a kit of parts for making a crosslinkable hydrogel composite. The kit of parts may comprise two parts, wherein a first part is a modified poloxamer having a first charge moiety and a second part is a peptide having a second charge moiety, wherein the first charge moiety and the second charge moiety are oppositely charged for ionic interaction between the modified poloxamer and the peptide. At least one of the modified poloxamer and the peptide comprises a crosslinkable moiety. Included in this description shall be a kit, wherein an initiator may be provided in either parts, or wherein the initiatior may be provided together with any of the two parts above. The parts may each be contained in a container, such as, for example, a syringe, a vial or a bottle. They may be contained undiluted, hence as a neat material, or in solution.

In a fourth aspect, there is provided a bioshaping method, the method comprising providing the hydrogel composite as described above, shaping the hydrogel composite to obtain a shaped hydrogel composite, and carrying out a cross-linking reaction on the shaped hydrogel composite to obtain a three-dimensional crosslinked network.

The term “bioshaping” as used herein refers to any process which moulds the thermo-responsive hydrogel composite as described above into a particular form, such as a particular three-dimensional object, being present in a semi-permanent state. Bioshaping may refer to biocasting or bioprinting. With the preamble “bio” is meant that this shaping may be undertaken at physiological conditions, such as at a mammal's body temperature, or even on the mammal's body. The mammal may be a human.

In various embodiments, the shaping step may be carried out by printing (or bioprinting). The printer may be able to print three-dimensional objects. The printing process may be performed by providing a first layer and adding multiple subsequent layers on top of the first layer, until a three-dimensional object is obtained. Any printer may be used as long as it provides a three-dimensional shape as discussed above.

In various embodiments, the printing may be carried out through a first nozzle of a printer. Advantageously, the hydrogel composites as described above may be printed without the use of a sacrificial support material. Hence, it may be possible to only use one nozzle in the bioprinting process.

In some embodiments, the method may further comprise dispensing a poloxamer through a second nozzle of the printer. This embodiment may be used in case more complex structures are desired to be printed. The poloxamer in the second nozzle may be a sacrificial material, hence, a material which is to be removed after the printing process and which mainly serves to provide stability during the printing process.

The poloxamer as used in the second nozzle may not be charged. Hence, it may not form the ionic interaction with the peptide and may thus be removed after the printing process.

The printing may be performed as a single printing step. This means that in a single printing step a three-dimensional object may be obtained. This may be called a “direct printing” process.

As an alternative to the above described bioprinting method, the bioshaping may be carried out by casting.

After the bioshaping step, crosslinking of the shaped hydrogel composite may be initiated. According to various embodiments, carrying out the crosslinking reaction on the shaped hydrogel composite may comprise irradiating the shaped hydrogel composite with electromagnetic radiation having a wavelength in the range of 10 nm to 1000 nm, preferably a wavelength of 10 nm to 400 nm, to obtain the three-dimensional crosslinked network. Carrying out the crosslinking reaction with electromagnetic radiation may be referred to as photo-initiated crosslinking. In these embodiments, the shaped hydrogel composite may be exposed to electromagnetic radiation.

The wavelength of the electromagnetic radiation may be in the range of UV wavelength, visible light wavelength or near IR wavelength. Hence, the wavelength may be in the range of about 10 to about 1000 nm, or in a range of about 10 to about 800 nm, or in a range of about 10 to about 600 nm, or in a range of about 10 to about 400 nm, or in a range of about 100 to about 500 nm, or in a range of about 200 to about 500 nm, or in a range of about 300 to about 400 nm. In preferred embodiments, the crosslinking may be performed through UV curing. Hence, in various embodiments, the crosslinking may be performed at a wavelength of 10 nm to 400 nm.

During this step, crosslinkable moieties on the peptide or the modified poloxamer may crosslink with one another, thereby increasing stability of the hydrogel composite by formation of covalent carbon-carbon bonds throughout the composite and resulting in a crosslinked network. Hence, this step would be referred to as a chemical crosslinking step. The crosslinking reaction may be a radical polymerization. It may also be referred to as a curing step. The radical polymerization may be initiated by a reactive species generated by the photoinitiator. As mentioned before, the exposure to electromagnetic radiation may cause the photo-crosslinkable moiety of the peptide to crosslink with each other by formation of carbon-carbon bonds. Hence, in various embodiments, the crosslinking reaction on the shaped hydrogel composite may comprise crosslinking the crosslinkable moiety of the peptide by formation of carbon-carbon bonds. This may mean that the crosslinkable moiety may be consumed after this step.

The method may further comprise a washing step to be performed after the crosslinking step. The washing step may be performed in order to remove any unreacted components from the obtained three-dimensional network.

In various embodiments, the washing step may comprise washing the obtained three-dimensional crosslinked network with a polar solvent such as alcohol or water. In one example, water was used in the washing step. The temperature of the water may be colder than room temperature, for example, the temperature of the water may be from 0 to about 15° C., or from 0 to about 10° C., or from 0 to about 5° C. The water may be referred to as “cold water”.

The bioshaping method may be carried out at a temperature of about 10° C. to about 50° C., or of about 20° C. to about 50° C., or of about 30° C. to about 50° C., or of about 10° C. to about 40° C., preferably at a temperature of about 20° C. to about 40° C., more preferably at a mammal's body temperature.

In a fifth aspect, a three-dimensional crosslinked network may be obtainable from a hydrogel composite as described above by using the bioshaping method as described above. Advantageously, the three-dimensional crosslinked network may be both biocompatible and biodegradable. Further advantageously, the three-dimensional crosslinked network obtained may be patent, thereby allowing for liquids or gases to pass through the three-dimensional crosslinked network. Further advantageously, the three-dimensional crosslinked network may be porous, which allows for a higher surface area. Further advantageously, the three-dimensional crosslinked network may be used as a platform for cell attachment and proliferation.

In a sixth aspect, there is provided a three-dimensional crosslinked network as described above for use in therapy. Due to the properties and characteristics of the three-dimensional crosslinked network as described herein, it may be possible to use the three-dimensional crosslinked network as a vascularization system by implanting the three-dimensional crosslinked network into a mammal's body. This may improve a medical condition wherein the mammal has a vascularization insufficiency. The implant may also be fabricated to function as an artificial organ. Thus, the implant may have a therapeutic effect.

In a seventh aspect, there is provided a method of treating vascularization insufficiency comprising administering to a mammal an effective amount of a three-dimensional crosslinked network as described above.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

Hereinafter, the present disclosure will be described more fully with reference to the accompanying drawings, in which exemplary embodiments of the invention are shown. This invention may, however, be embodied in many different forms and should not be construed as limited to the exemplary embodiments set forth herein. Rather, these embodiments are provided so that this disclosure will be thorough and complete, and will fully convey the scope of the invention to those skilled in the art. In the drawings, lengths and sizes of layers and regions may be exaggerated for clarity.

### Experimental Section

The bio-ink hydrogel disclosed in here is able to overcome the before mentioned problems. This material has three distinct features which are (1) high printability at room and human body temperatures; (2) high mechanical strength and (3) higher porosity. With the present disclosure, complex biological structures for biomedical applications can be designed and printed. Moreover, after being crosslinked, this hydrogel has sufficient mechanical strength to be handled and can be placed in a bioreactor for further tissue maturation in vitro before implantation. The present disclosure is made possible by combining two highly printable hydrogels—pluronic-GelMA composite and pluronic, in a certain proportion and sequence. With this disclosure, free standing 3D shapes can be printed which is very unique and different from other bioprinting techniques found in the literature.

Advantages and Improvements Over Existing Methods, Devices or Materials

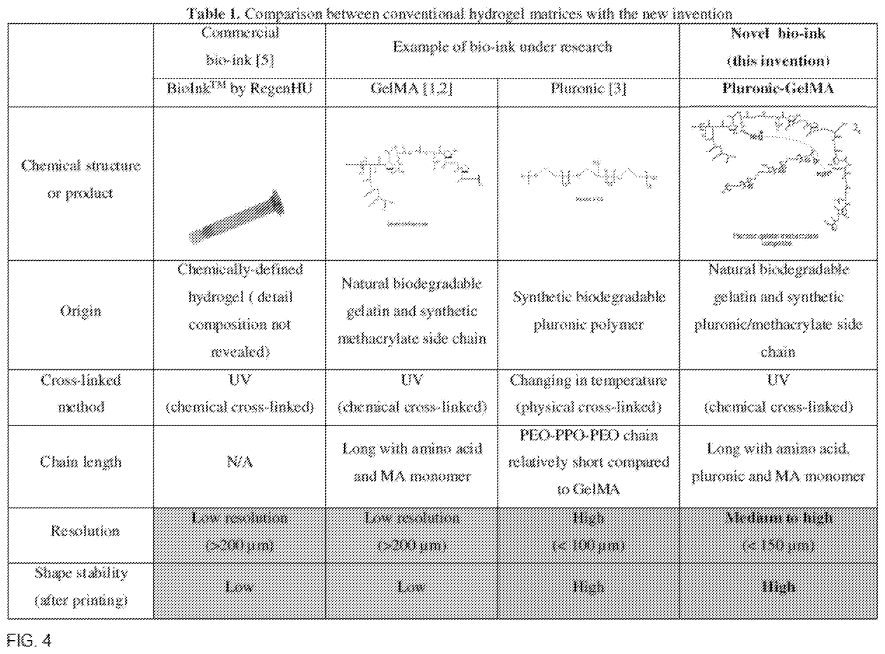

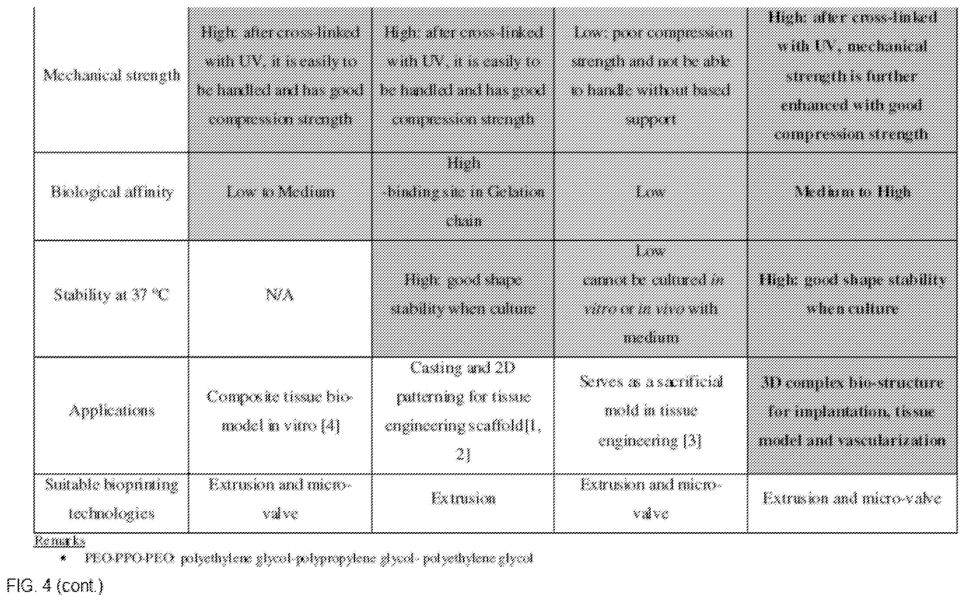

Important aspects of this disclosure in bio-ink are pluronic F127 and gelatin methacrylate, where both are biodegradable and biocompatible. They have been used widely for tissue engineering and other life science-related applications. The main advantage of this bio-ink is in its superior printability performance. It can be processed and printed into a shape of high complexity without clogging the print head for ease of operation. After curing with UV light, the printed structures will have relatively higher mechanical properties compared to other types of hydrogel materials (as shown in the table provided as FIG. 4). This bio-ink also has good biological affinity and tuneable degradation rate, which allows expanding the applications of 3D bioprinting parts from in situ implant to in vitro toxicity test. Hence, this hydrogel concept will create a valuable bio-matrix that can lead to advancement in biomedical research.

The hydrogel not only achieves printability at room temperature, it can be printed at human body temperature, which is the optimum temperature for cell growth and proliferation. This ensures that the hydrogel constructs can be printed together with cells in this environment. In addition, the hydrogel's high porosity is good for nutrient and oxygen diffusion which is an essential property for tissue engineering. The higher porosity will promote tissue growth and provide better cell-cell interaction. Thus, this hydrogel is also suitable to serve as tissue engineering scaffold.

As disclosed herein, a new hydrogel composite that provides printability, shape integrity and biocompatibility for fabricating complex 3D structures in a single step without relying on any liquid platform (e.g. gelatin slurry and CaCl₂ solution) is provided. The highly printable hydrogel composite was designed and fabricated from Pluronic 127 and GelMA. After that, the hydrogel composite was printed along with Pluronic 127 to achieve a perfusable vasculature-like structure. Rheological properties, water swelling properties, the cytotoxicity and cell differentiation of this hydrogel composite were evaluated by using L929 fibroblasts and human umbilical vein endothelial cells (HUVECs). The results show that the combined use of the hydrogel composite and Pluronic offers a surprising capability of freeform printing of biocompatible hydrogels.

The 3D complex hollow structures are desirable for many applications, especially for vascularized tissue engineering such as larger veins which have a vessel wall thickness around 500 μm or even tissue models for drug testing. Nowadays, limited options are available for fabricating soft 3D complex hollow structures directly on a solid platform in a single step. The new hydrogel composite developed in this work has good printability, shape integrity and biocompatibility. The combination of a sacrificial material with this new hydrogel composite allows a single step printing of soft and perfusable vasculature-like structures. Therefore, this work presents a great enabling potential for many tissue engineering applications. Furthermore, by integrating the new hydrogel composite with other emerging techniques, such as 4D bioprinting and in vivo bioprinting, tissue engineering would be further advanced in the near future with more potential in upscale printing of 3D complex structures.

In various embodiments, a series of hydrogel composites was synthesized. The hydrogel composites were composed of a carboxylated pluronic 127 and a methacrylate-modified gelatin. The viscosity, shear rate and water swelling ratio of the ensuing hydrogel composites were investigated. The thermoresponsive and crosslinkable properties of the hydrogel composites were demonstrated by bioprinting the hydrogel composite into various shapes and crosslinking the obtained three-dimensional shape. Finally, the three-dimensional networks obtainable by the bioprinting process were tested for cell viability and cell proliferation.

Discussion

According to embodiments disclosed herein, a highly printable and biocompatible hydrogel composite was successfully developed. By modifying Pluronic into Plu-MP, the hydrogel composite is able to stay in single phase due to the physical ionic bond between carboxylic group and amine group. Moreover, by using a three-way stopcock, the energy was added into the mixture similar to the use of homogenizer. The energy that was added into the system might lead to the formation of stable foam structure inside the hydrogel.

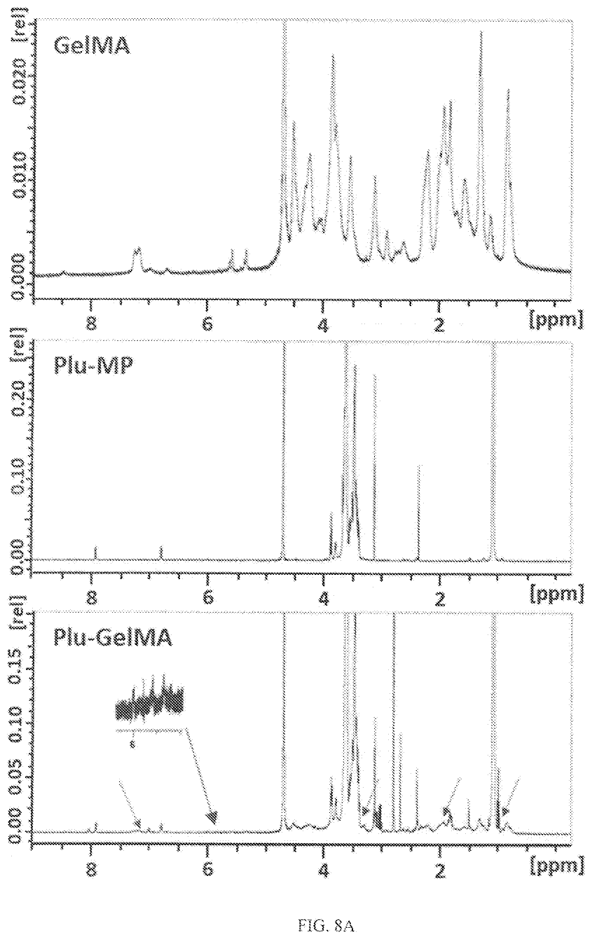

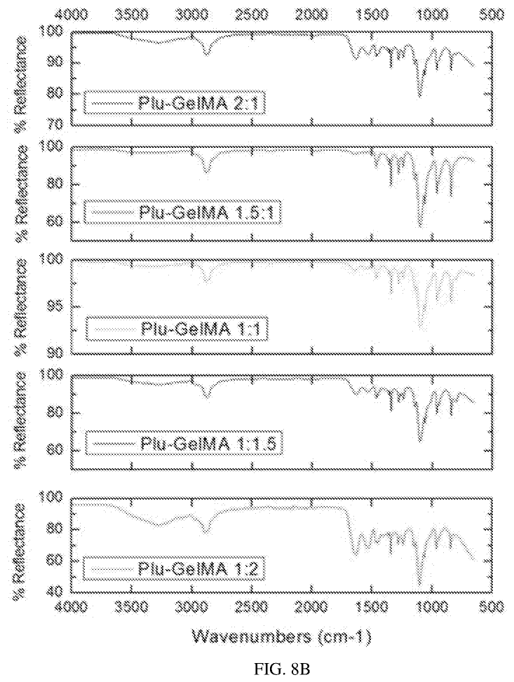

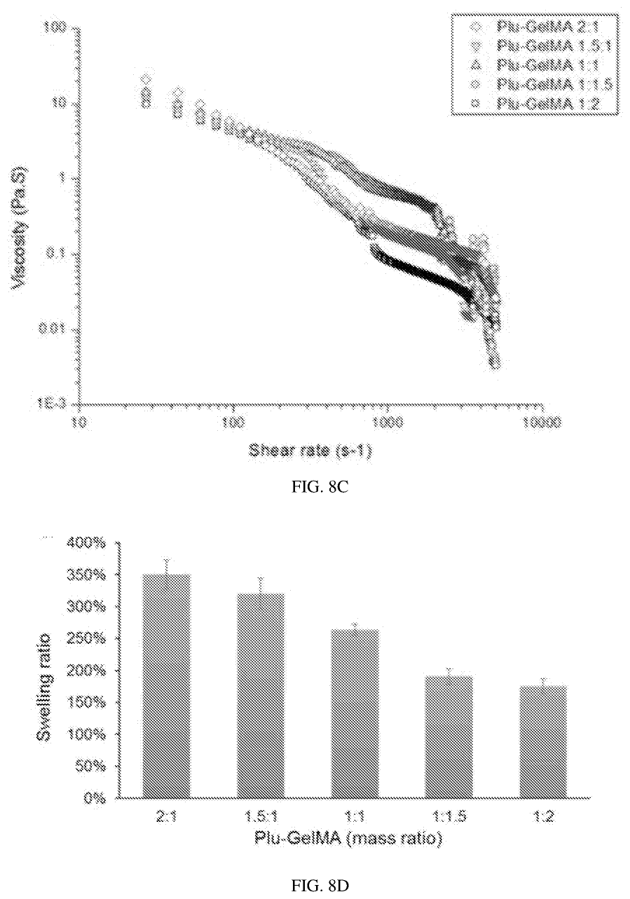

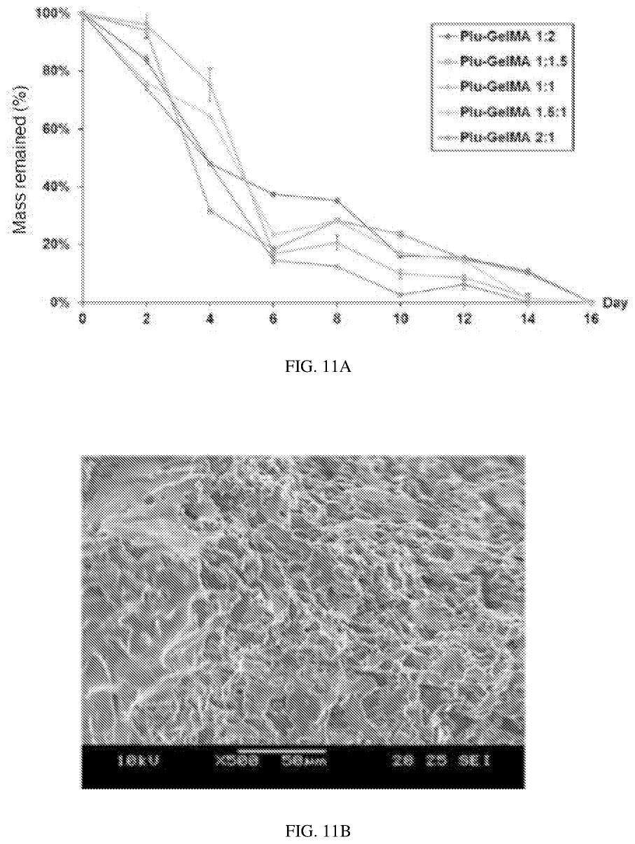

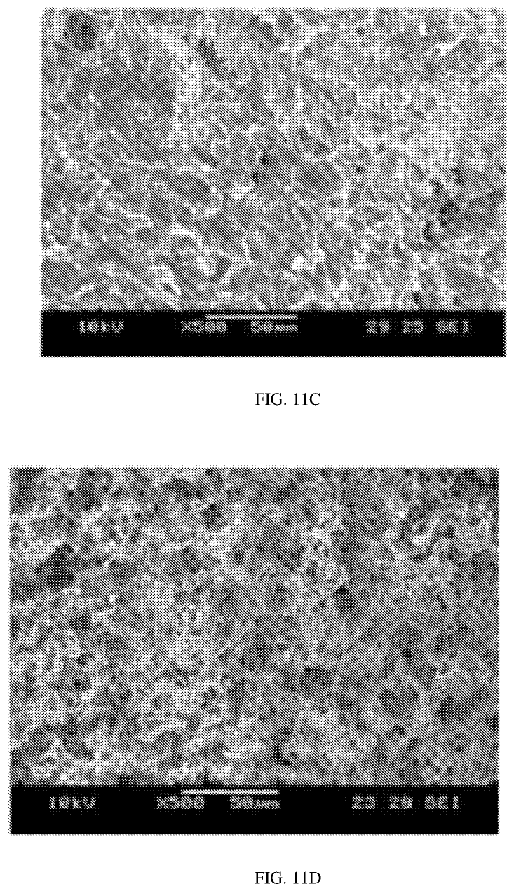

The UV crosslinking process of the GelMA improves the shape integrity of the printed structures. However, unlike the grafting reaction by EDC/NHS coupling, by using this technique, some of the Pluronic did not react but bind with GelMA. Thus, the use of cold water was required in order to eliminate unreacted part which might affect cell viability during cell culture. This is because Pluronic is not able to provide mechanical strength to support cell adhesion due to the reversible physical gelation. Moreover, due to different starting concentrations (based on gelation concentration), the higher mass ratio of GelMA leads to the presence of more free water groups as shown earlier in FIG. 8B. As expected, the GelMA contents in the composite affected water swelling properties, and higher GelMA contents led to lower water absorbability (FIG. 8D). Additionally, the strong covalent bonds formed from photocrosslinking reaction might also contribute to lower swelling ratio. Moreover, when there was more Pluronic in the composite (e.g. Plu-GelMA 2:1), the pore structure was larger (as shown in FIG. 11D). This might also contribute to the swelling ratio as a larger pore structure allows more water to be absorbed.

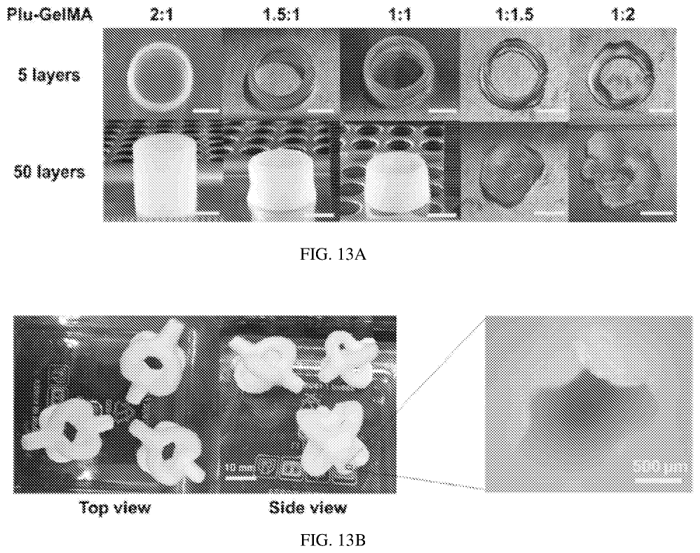

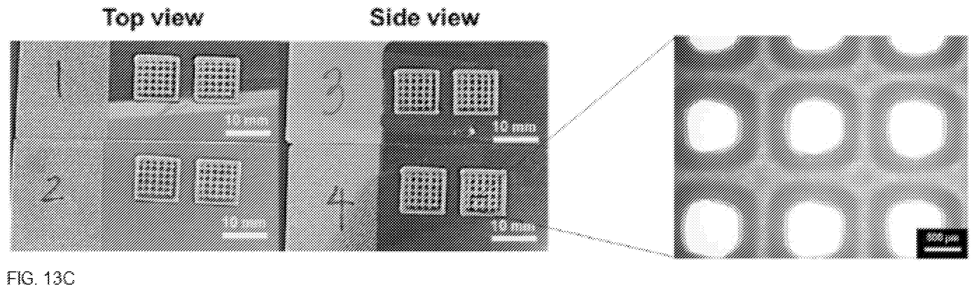

The printability of Plu-GelMA is dependent on Pluronic contents, the more Pluronic, the better the printability, as shown in the FIG. 13A to FIG. 13C. This is because Pluronic is packed in micelles, which is easily flowable and structurally stable at a wide range of temperatures (from 20° C.-40° C.). Therefore, the Plu-GelMA 2:1 was selected to be the model material for printing 3D complex structures. Advantageously, by keeping the mass ratio to 2:1 and not increasing the ratio of the Pluronic, UV curing may be improved. Moreover, Plu-GelMA 2:1 is stable at a wide range of temperature similar to Pluronic, thus printing at cell favourable environment such as human body temperature is possible. It is recommended to keep the concentration of Pluronic in model material and support material close enough to each other to avoid material interaction and diffusion, due to the differences in osmotic pressure. The 3D quadfurcated vasculature-like structure was fabricated by using Plu-GelMA 2:1 as model material and 24.5 wt % Pluronic as support material on a solid platform in a single step printing. The liquid and gas perfusion test proved that the 3D structure was patent and perfusable. However, some of the liquid ink was absorbed into the structure at the initial stages of the test due to swelling and porosity of the hydrogel.

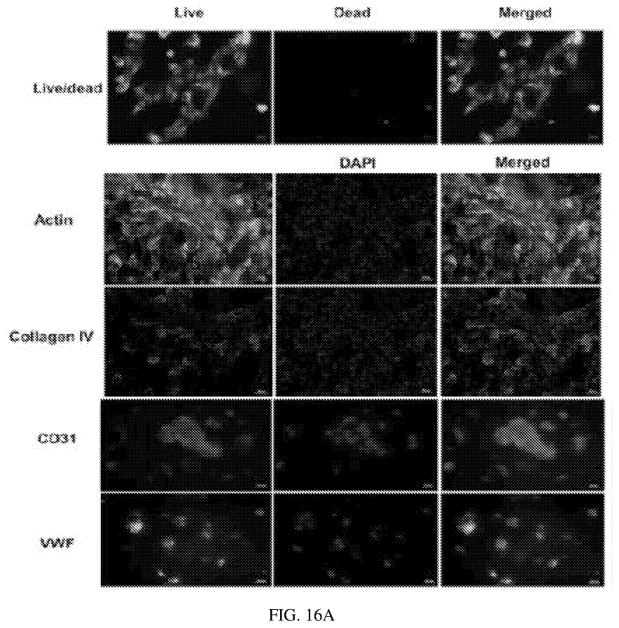

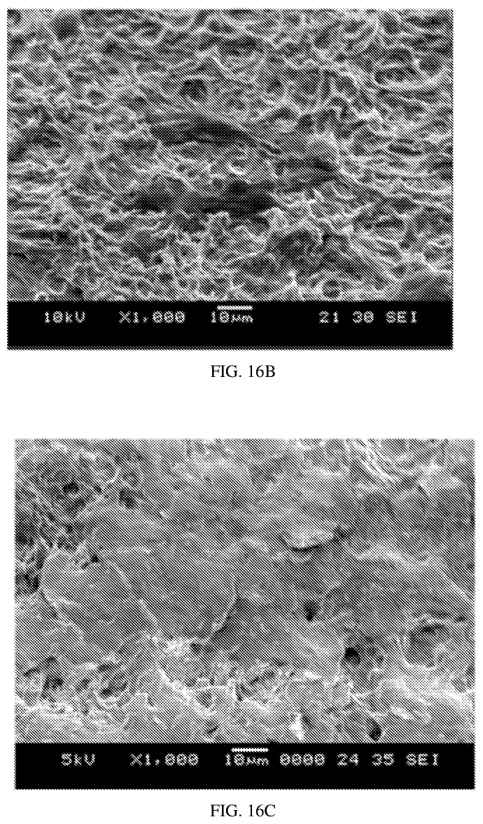

From in vitro evaluation, L929 cells were alive and proliferated over 7 days on all composites. The Plu-GelMA 2:1 achieved the highest cell number compared to other ratios, perhaps due to higher swelling ratio and larger pores in microstructure. The larger pores can lead to more surface area and better medium transportation through the entire structure. The L929 cells were able to attach onto the hydrogel composites and able to pack together when the cell number increased. This proved that the Plu-GelMA 2:1 hydrogel composite provides a good platform for cell attachment and proliferation. On the other hand, the results of HUVECs study proved that the Plu-GelMA 2:1 hydrogel composite supported differentiated cells, as evidenced by the expression of two markers of endothelium cells—CD31 and VWF. CD31 shows the formation and fusing of HUVECs which may lead to the angiogenesis and vascular branching if the cell culture is continued. The presence of VWF shows the efficiency of vascularization. Lastly, as shown in FIG. 16B, primary HUVECs could also attach and spread on the composite surface. At day 7, when the number of cells was sufficient, the HUVECs fused and covered the hydrogel composite surface with their extracellular matrices, which confirmed that this hydrogel composite was able to support attachment for different types of cells.

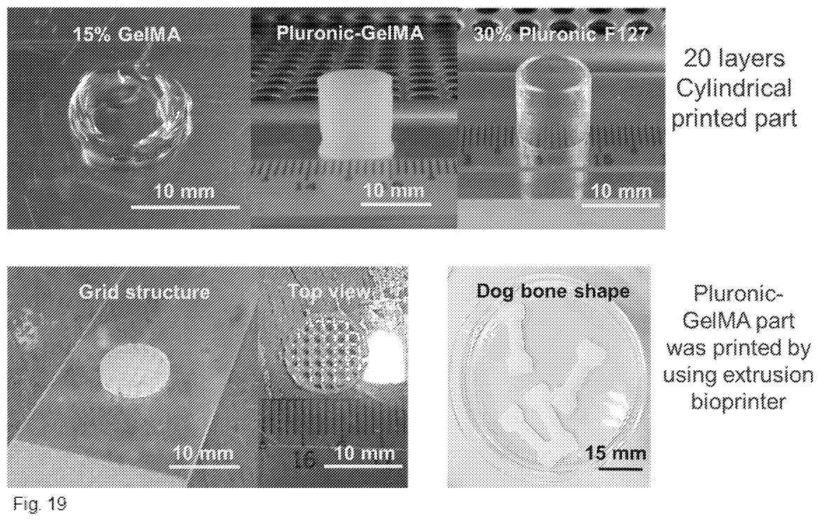

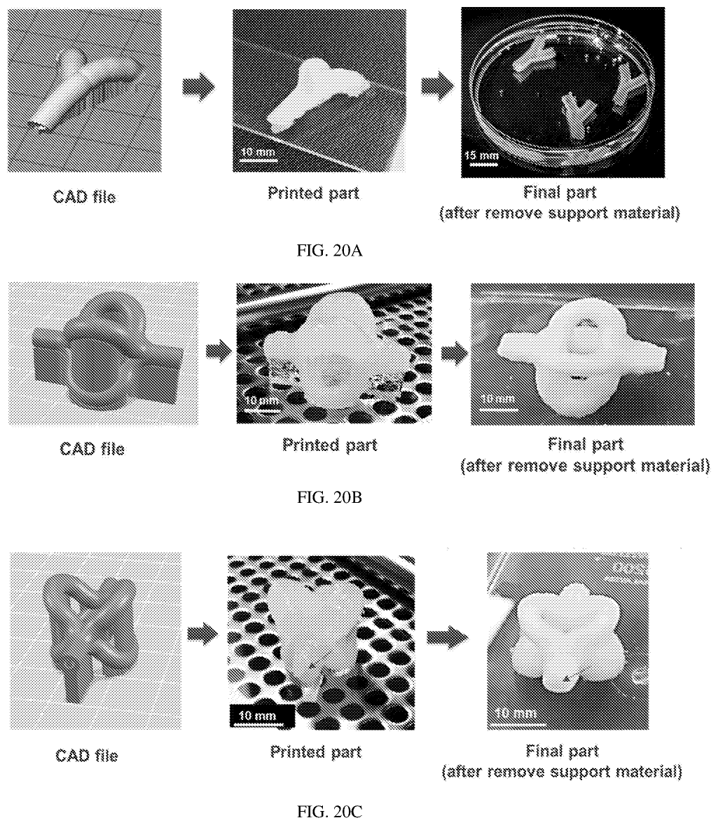

The inventors were able to demonstrate the superior printability properties of the bio-ink hydrogel (Pluronic-GelMA) by comparing it to Pluronic F127 (see FIG. 19). For synthesizing an overhanging 3D complex structure (FIG. 20A to FIG. 20C), Pluronic-GelMA is the model material while pluronic F127 is support material. After printing, the printed part has to be cured for 120 seconds (Pluronic-GelMA contains photo-initiator inside which was added prior printing). Lastly, pluronic, the support material, was removed by using cold water. The results of printing (final part) are shown in FIG. 20A to FIG. 20C.

### EXAMPLE 1

### Synthesis Process of Pluronic-GelMA Composite

The pluronic chain of pluronic 127 was modified by reaction with succinic anhydride to form pluronic monocarboxylate (Plu-MP) similar to the method described by Park et al. Firstly, Pluronic F127 was dissolved in dioxane. In this reaction, 4-dimethylaminopyridine (DMAP) and triethanolamine (TEA) were used as a catalyst or an activator. The reaction was performed under vigorous stirring condition for 24 hours. Next, the dioxane was removed and the Plu-MP powder was precipitated by washing with diethyl-ether, before being dried in a vacuum oven overnight to remove the organic volatile compounds. Then, purified Plu-MP powder was mixed with PBS to make 30 wt % Plu-MP hydrogel. The hydrogel was later stored at 4° C. before further synthesis and testing. Gelatin methacrylate (GelMA) was fabricated by reaction between methacrylate anhydride and gelatin at 50° C. in phosphate buffer saline (PBS), similar to the method previously described by Narbat et al. The reaction was run for two hours under constant stirring. Afterwards, the reaction was stopped by diluting the solution fivefold with PBS. The diluted solution was further dialyzed with deionized water by using a 12-14 kDa molecular weight cutoff (MWCO) dialysis tube for one week. Subsequently, the GelMA was frozen overnight at −30° C., lyophilized for 5-7 days, and stored at −30° C. GelMA was prepared by mixing freeze-dried GelMA foam at concentrations of 15 wt % and 0.2 wt % of the photo-initiator (2-Hydroxy-4′-(2-hydroxyethoxy)-2-methylpropiophenone, Irgacure 2959) in PBS. GelMA with photo-initiator was kept in the dark at 37° C. before mixing to prevent gelation.

Finally, Plu-MP was mixed with GelMA by placing the two components into two separate syringes; 15 w/v % GelMA with 0.2 w/v % of Irgacure 2959 and 30 w/v % Plu-MP at varying mass ratios. Then, the two syringes were connected using a 3-way stopcock (Discofix® C, B. Braun). The materials in both syringes were mixed by transferring the two hydrogels into the respective other syringe until they turn into one homogeneous hydrogel. After that, the mixed hydrogel was kept for overnight at room temperature in the dark to allow the reaction to finish completely. The hydrogel composites at different mass ratios of Plu-MP:GelMA were casted or printed in different shapes followed by UV crosslinking for 120 seconds by using UV flood curing system (Techno Digm, Singapore) before further characterization (except NMR). All the chemicals in this work were purchased from Sigma-Aldrich unless mentioned elsewhere.

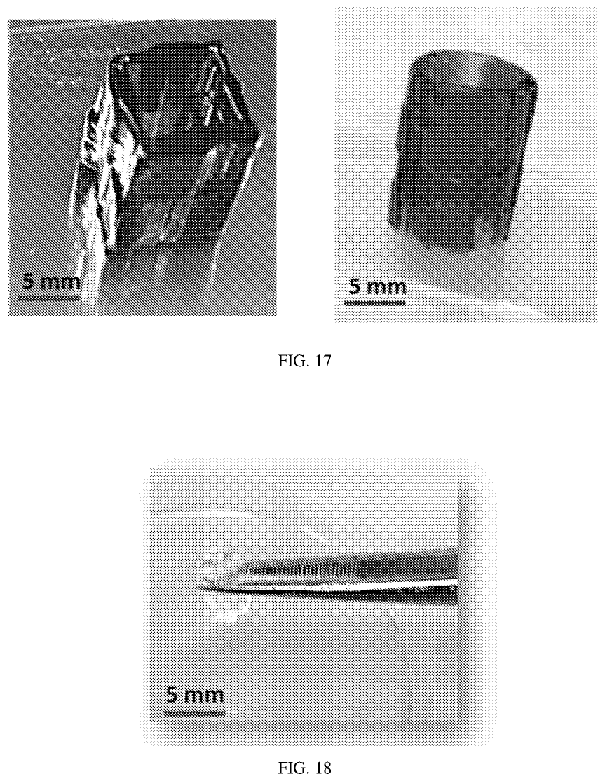

In this study, two materials, namely Pluronic F127 and Gelatin Methacrylate were used in synthesizing a new product. Pluronic F127 is a thermo-reversible hydrogel which has high printability and biocompatible. However, pluronic is a physical cross-linked hydrogel (non-permanent bond), it is not strong enough to be handled in vitro (FIG. 17).

On the other hand, Gelatin methacrylate (GelMA) is a UV cross-linked hydrogel which has high mechanical strength (after curing; as shown in FIG. 18) and bioactive property. Nonetheless, before curing, GelMA has very low printability so it is normally used for casting with different types of mold.

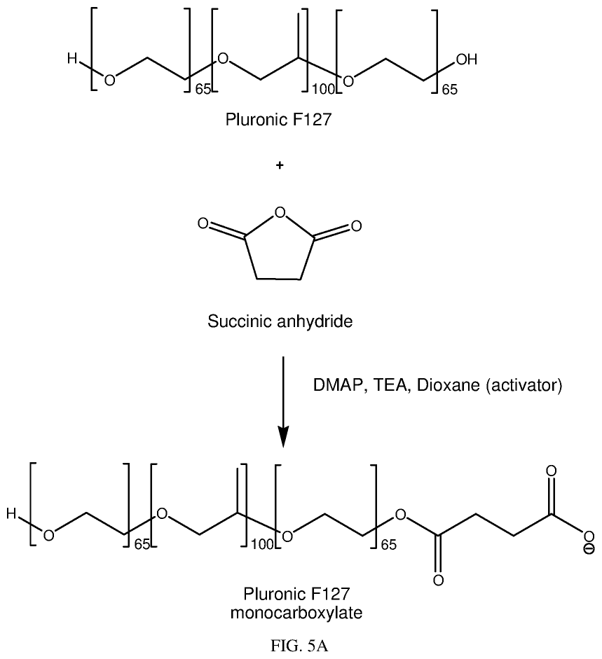

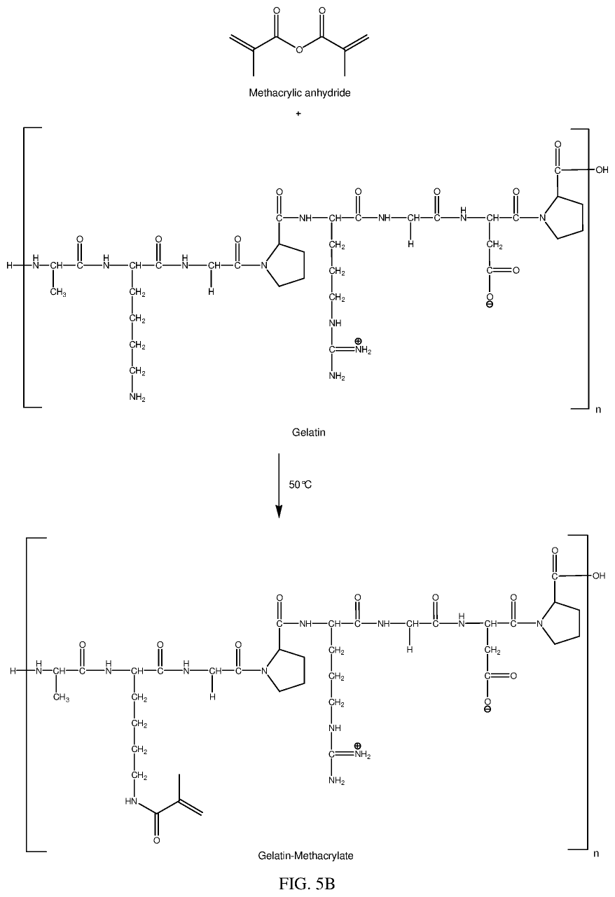

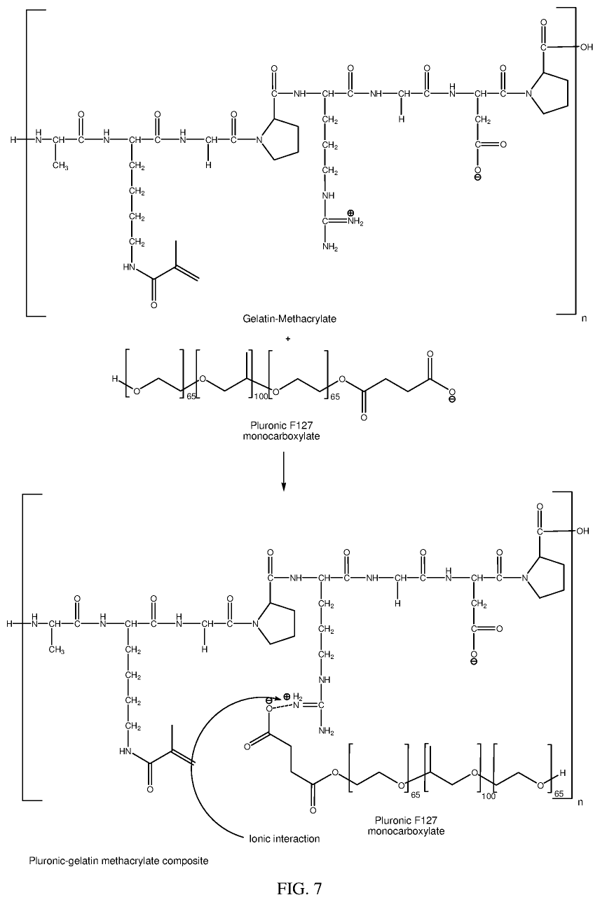

The combinatory use of both pluronic monocarboxylate and GelMA will yield a high performance matrix for bioprinting (gelatin-pluronic/MA). As illustrated in FIGS. 5A and 5B, the pluronic chain was modified by reaction with succinic anhydride to form pluronic monocarboxylate (PluMP). In this reaction, 4-dimethylaminopyridine (DMAP), triethanolamine (TEA) and 1,4 dioxane were required to act as a catalyst or an activator. GelMA was fabricated by reaction between methacrylate anhydride and gelatin at 50° C. Finally, as demonstrated in FIG. 7, pluronic monocarboxylate was ionically connected “as side chains of GelMA”, which initially involved placing both components separately into syringe; 15% GelMA with 0.2% of photo-initiator (Irgacure 2959) and 30% PluMP at ratio 1:1 as shown in Table 1. Before mixing, GelMA with the photo-initiator was kept in the dark at 37° C. before mixing to prevent gelation. On the other hand, PluMP was kept at 4° C. before mixing. Then, the two syringes were connected using 3-ways stop cocks. The materials in both syringes were mixed by putting two hydrogels into one another syringe until it turns into a homogeneous hydrogel. After that, the mixed hydrogel was kept overnight at room temperature in the dark to allow the reaction to finish completely.

TABLE 1
Composition of Bio-ink before mixing

| | Reagent | Concentration | |
|---|---|---|---|
| Syringe 1 | Gelatin methacrylate | 15% | w/v |
| | (GelMA) | | |
| | Photo initiator: | 0.2% | w/v |
| | Irgacure 2959 | | |
| Syringe 2 | Pluronic | 30% | w/v |
| | Monocarboxylate | | |
| | (PluMP) | | |

As mentioned above, the synthesis started with Plu-MP and GelMA hydrogel (chemical structure of both compounds are shown in FIG. 5A and FIG. 5B) in liquid state at different mass ratios (Plu-MP:GelMA=1:2, 1:1.5, 1:1, 1.5:1 and 2:1) by using a 3-way stopcock connector. As shown in FIG. 6B, the reaction started upon mixing Plu-MP micelles with GelMA un-coiled chains to form a weak physical bond between —COO⁻ group and —C(NH₂⁺)NH₂ and to provide good stability (as shown in FIG. 2A and FIG. 2B, FIG. 6B and FIG. 7). After mixing, the samples were kept overnight, and after keeping at room temperature, the hydrogel was allowed to completely shift from solution (liquid) state into homogeneous and translucent gel (solid) state in the macro level. Next as shown in FIG. 6B, the hydrogel composite was used for casting or printing, followed by the UV exposure to generate photo-crosslinked chemical bonds in the hydrogel structure. The unreacted Plu-MP was later washed away by using cold water.

### EXAMPLE 2

### Characterization of Pluronic-GelMA (Plu-GelMA) Hydrogel Composite

Chemical Properties: Fourier Transform Infrared (FTIR) Spectroscopy

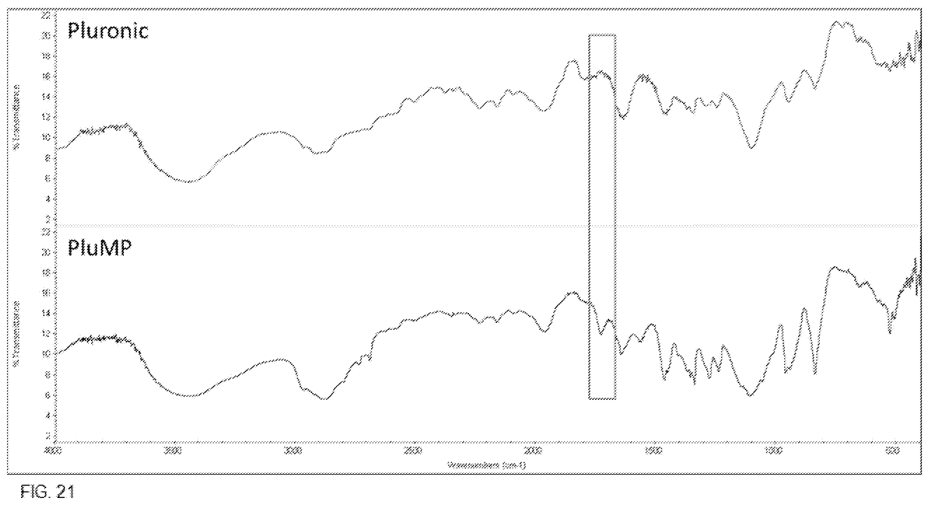

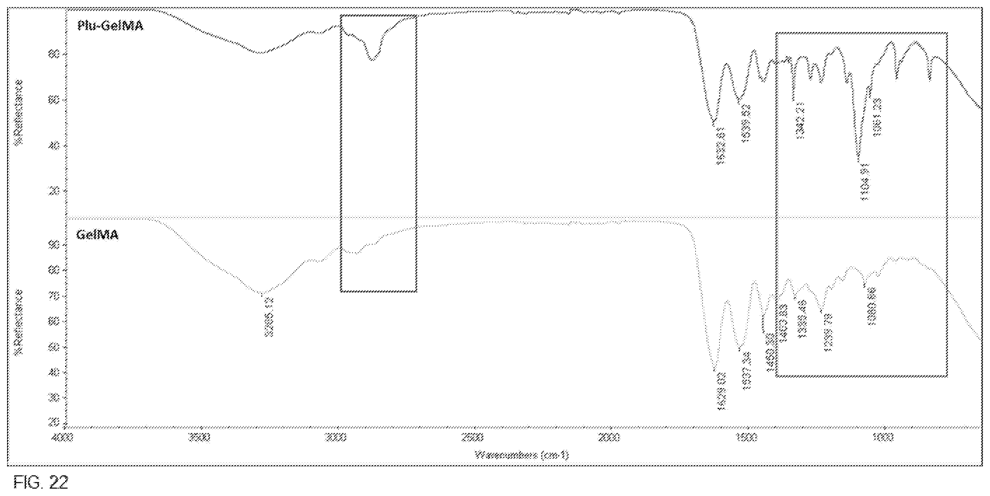

The FTIR curve confirmed that there was a change in chemical structure between PluMP and pluronic. This is shown in the C═O bond for carboxylic acids, which may be observed at 1720-1706 cm⁻¹ as shown in FIG. 21.

The FTIR curve confirmed the formation of a pluronic-GelMA composite by comparing the FTIR of FIG. 22 with the previous FTIR of the original reactant (GelMA), as shown in FIG. 22.

Chemical Properties: Nuclear Magnetic Resonance Spectroscopy (NMR)

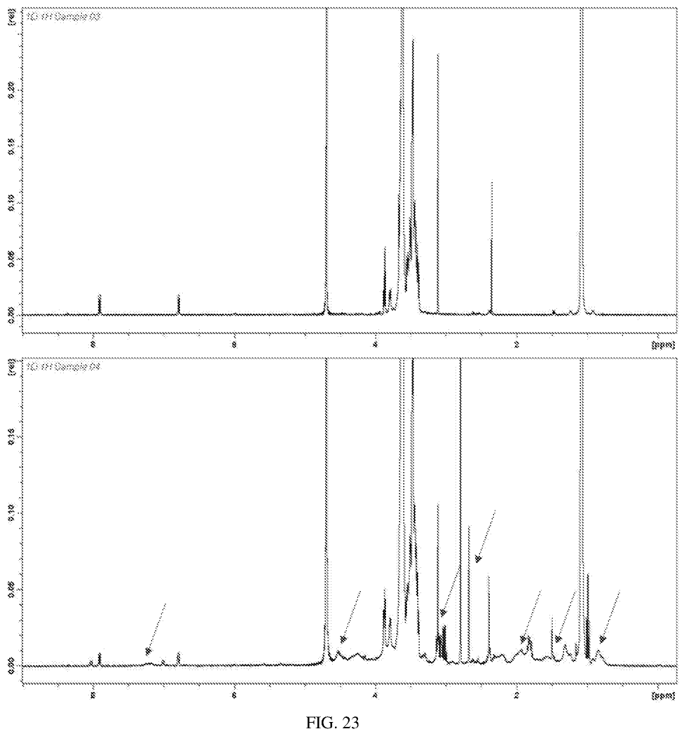

¹H NMR (400 MHz) results confirmed that there are changes in the chemical fingerprint between PluMP and pluronic-GelMA as pointed out by arrow in FIG. 23.

For ¹H-Nuclear magnetic resonance spectroscopy (¹H-NMR), the reactants and synthesized polymers were characterized by 1H-NMR 400 MHz, (AVANCE I, Bruker, Germany) in D₂O solvent (Sigma-Aldrich, USA) wherein the peaks at 1.8-2.0, 5.7-5.9, 6.1-6.3 ppm where attributed to the methacrylate group and the peaks at 1.0-1.2, 3.5-3.7 ppm to the Plu-MP functional group. For attenuated total reflectance, Fourier transform infrared spectroscopy (ATR-FTIR), UV-crosslinked hydrogel samples were characterized in ATR mode by using Thermo Scientific Nicolet™ 6700 FT-IR spectrometer (Cambridge, UK) which was equipped with OMNIC software. Samples were mounted onto the orbit sampler. The spectra's results were demonstrated in the range of 500-4000 cm⁻¹ with a resolution of 4 cm⁻¹.

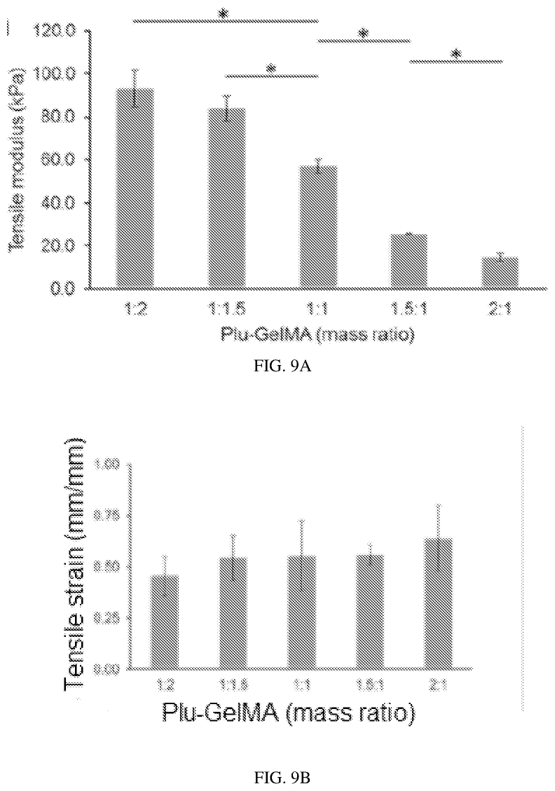

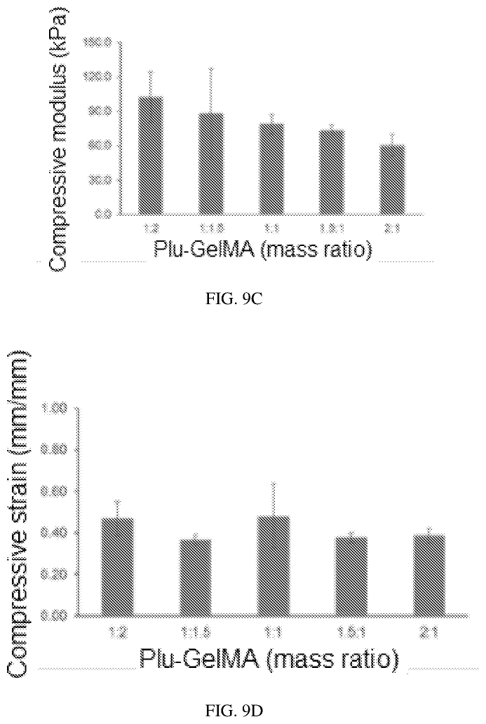

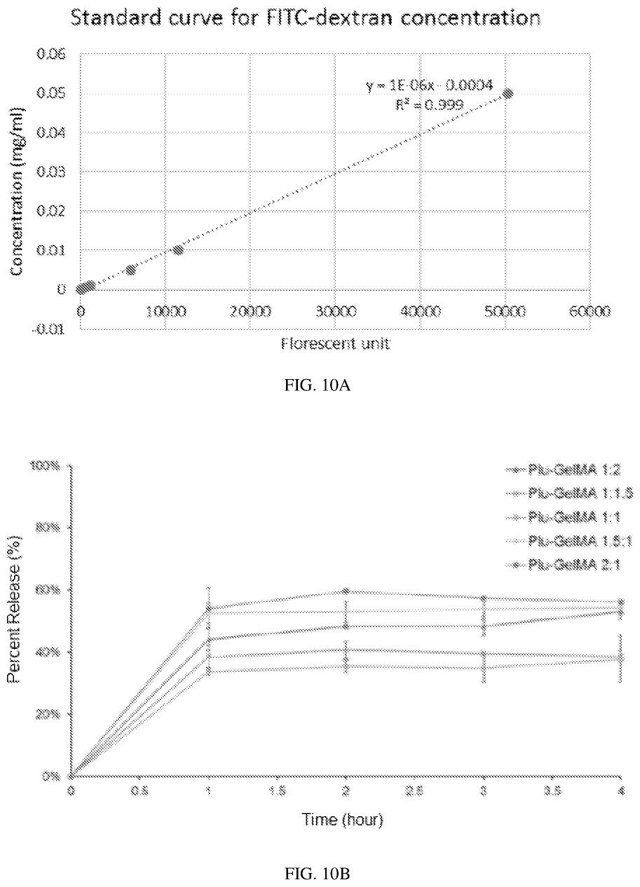

The NMR results showed that Plu-MP and GelMA were different from their composite Plu-GelMA due to the mixing and overnight reaction. The zoom-in image in FIG. 8A showed that the methacrylate functional group was present in Plu-GelMA, making the thermo-responsive composite also photo-crosslinkable. The FTIR results in FIG. 8B confirmed that more GelMA contents can lead to more hydrophilic O—H functional groups (free water hydroxyl groups) in 3200-3600 cm⁻¹, hence this would result in a higher water swelling ratio (FIG. 8D). The rheological properties of Plu-GelMA hydrogel composite were shown in FIG. 8C. All the hydrogels showed a shear thinning behaviour which was similar to Pluronic and GelMA. At the high shear rate region (shear rate beyond 100 s⁻¹), the viscosities of all hydrogel composites dropped drastically. At a higher content of Pluronic, especially at the ratio of 2:1, the overall shape of the curve looked more linear than at other ratios. Other properties such as mechanical modulus, diffusibility, enzymatic degradation and morphology were also investigated, however, the results showed that different mass ratios affected tensile modulus and microstructure only (FIG. 9A to 9D, FIG. 10A and 10B and FIG. 11A to 11D).

### EXAMPLE 3

### Effect of Phase Separation

As shown in FIG. 2A, the Pluronic-GelMA composite without the carboxyl group on the poloxamer, after 5-7 days, would become two phases and would lead to defects in the printed part as shown in FIG. 2B. Moreover, as the phases separated, there would be less GelMA contents which would lead to lower curability. Thus, there was a significant difference between carboxylated PluMP:GelMA and non-carboxylated Pluronic:GelMA, based on phase stability and curability.

### EXAMPLE 4

### Printing of Soft and Perfusable Vasculature-Like Structure

G-Code Generation

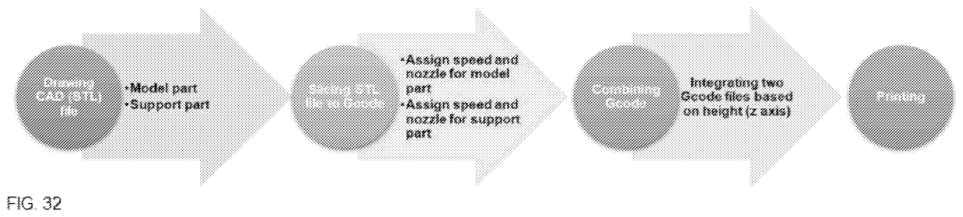

In order to print 3D hollow structure, two CAD files or STL files needed to be generated: one for the model part and another one for the support part. After that, each STL file was loaded into the STL converter program which was attached to a RegenHU bioprinter. This program changed each STL file into a G-code. In this step, stage printing speed, layer thickness as well as a specific nozzle needed to be assigned. After obtaining two G-code files, they were combined by using Matlab code which assigned the step based on the height of the structure (in z axis direction). After combining, the new G-code was ready to be used for printing. The summary of the overall process is shown in FIG. 32.

Printability Test and Printing of 3D Complex Structure

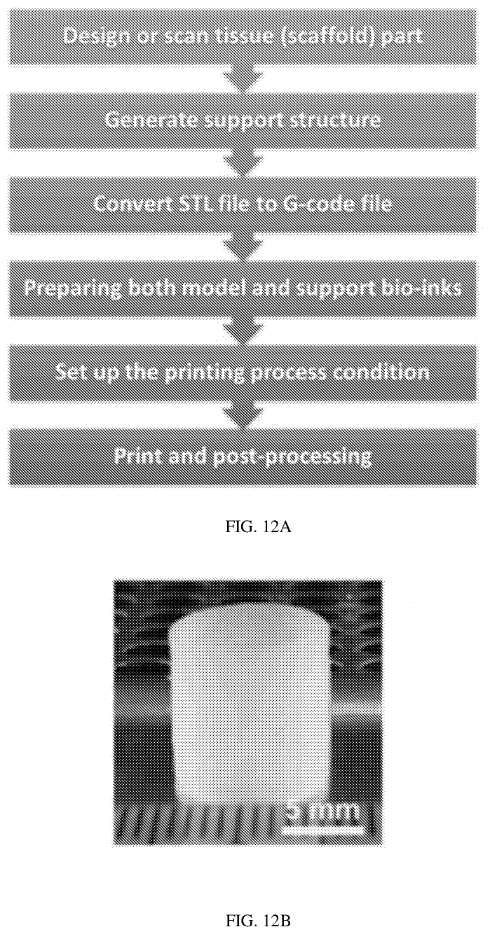

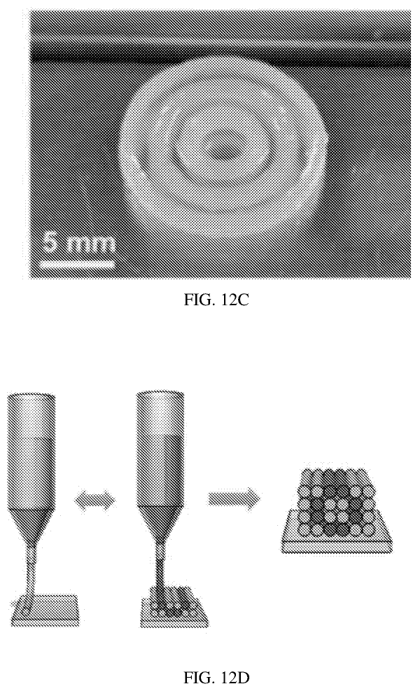

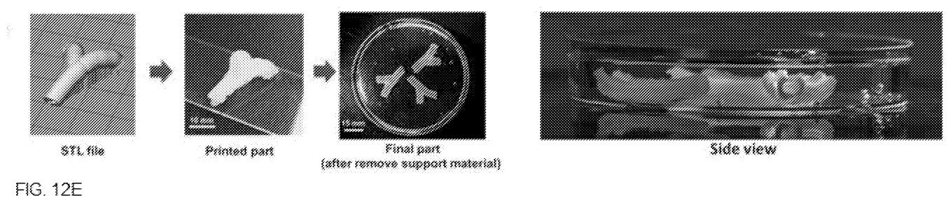

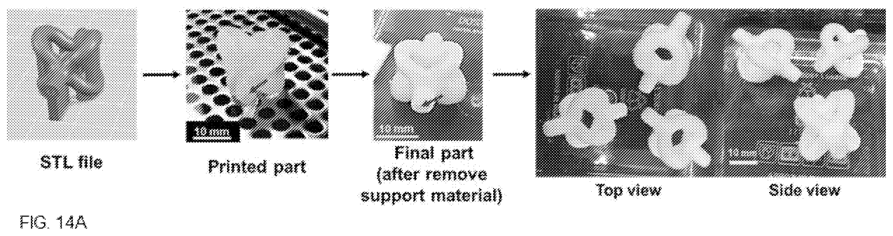

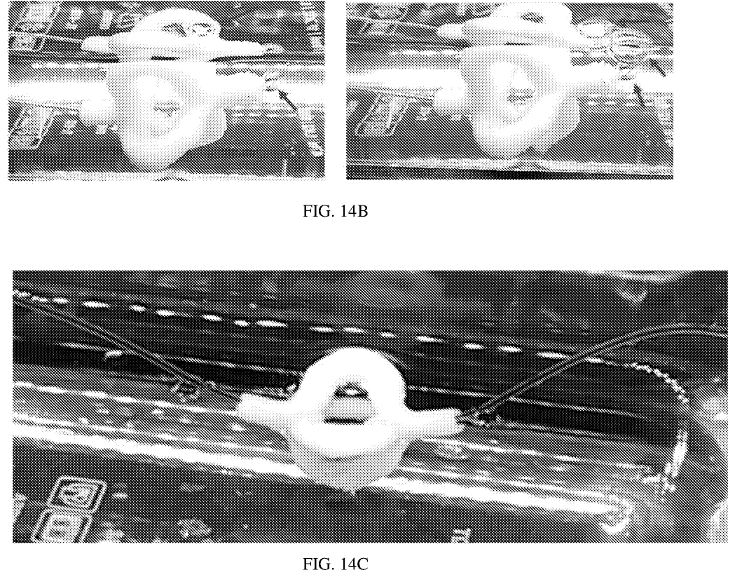

The cylindrical CAD file was designed by using BioCAD™. The STL files (as shown in FIGS. 12E and 14A) were sliced by using the STL converter program. Both programs were attached with a pneumatic extrusion-based bioprinter (Regenhu, Villaz-St-Pierre, Switzerland) and as mentioned above, overall G-code generation process was described in FIG. 32. The hydrogel composites were loaded in 5 ml syringe before printing. The printing condition of all concentrations was at stage moving speed of 500 mm/min, pressure 3-5 bar (depends on the ratio), at a temperature of about 30±3° C. and 27 G nozzle. 24.5% Pluronic F127 was used as support material. After printing, the hydrogel samples were cured by using UV flood curing system (Techno Digm, Singapore) for 120 seconds and soaked in cold water for 2-3 hours before undertaking further experiments. Later the hollow hydrogel structures were taken for perfusion tests using an air and a red dyed liquid to further investigate the perfusion property.

Printability and Repeatability

The comparison of printability of different mass ratio of Plu-GelMA showed that the 2:1 ratio provided the best performance at the high layer constructs (FIG. 13A). The 3D quadfurcated structures were also printed with three replicates as well as square grid shapes that were printed with eight replicates, as shown in FIGS. 13B and 13C, respectively. This shows that the 2:1 Plu-GelMA composite provided good printability and was able to generate good repeatability for both 2.5D and 3D constructs.

Moreover, the smallest feature sizes were shown in FIGS. 13B and 13C, where the smallest hole and the smallest grid line that can be printed with a 27 G nozzle was approximately 500 μm. Based on the pressure that can be used with the bioprinter with this material, the smallest nozzle that can be used is 32 G, which would make the resolution better and could be up to around 300 μm.

The printing process flow diagram is shown in FIG. 12A to FIG. 12E. For the simple hollow cylindrical structure (50 layers) and the multilayer, structure as shown in FIGS. 12B and 12C, support material was not required. Plu-GelMA at a ratio of 2:1 was used directly for single-step printing of these simple structures. Hydrogel composites at other mass ratios were also able to print but at a lower height only (less than 50 layers) as shown in FIG. 13A to FIG. 13C. In order to fabricate complex structures, support material and dual-nozzle printing may be used. The printing process is shown in FIG. 12D, in which one nozzle was used to print Plu-GelMA as model material and the other nozzle was used to print Pluronic as support material. Both model and support materials were printed using the same processing parameters, and good printability and repeatability were achieved (FIG. 12E). After printing, the parts were UV cured and washed in cold water to remove support materials. The patency of the hollow structure could be easily observed when the parts were immersed in the water (FIG. 12E).

In addition to Y-shaped vasculature, a 3D quadfurcated structure (FIG. 14A to FIG. 14C) could also be fabricated within 30 minutes, which is a similar printing speed compared to the extrusion based 3D bioprinter, when printing on a liquid platform. However, the presently described process did not need pre-processing of liquid bath platform, therefore the overall processing time was much shorter. As further discussed below, in order to test the perfusability of the hollow structure, air was first purged into the structure, followed by a red liquid perfusion test. Air bubbles were seen continuously exiting from the opening. Likewise, a moving stream of red liquid was clearly visible at the exit of the quadfurcated structure from another outlet tube. However, when the outlet tube was moved and became loose, the liquid was able to purge out directly from the exit of the 3D quadfurcated structure. FIGS. 14B and 14C show that this soft vasculature-like structure was perusable to both liquid and gas.

### EXAMPLE 5

### Mechanical Tests

Rheological Testing

A rheological study was conducted with each concentration of Plu-GelMA hydrogel composite by using a 40-mm, parallel plate rheometer (DHR, TA Instruments). The temperature was kept constant at room temperature throughout the experiment. The experiment was run at shear rate 0-5000 s⁻¹ in order to obtain a rheological profile of the hydrogel composite.

Rheological Properties

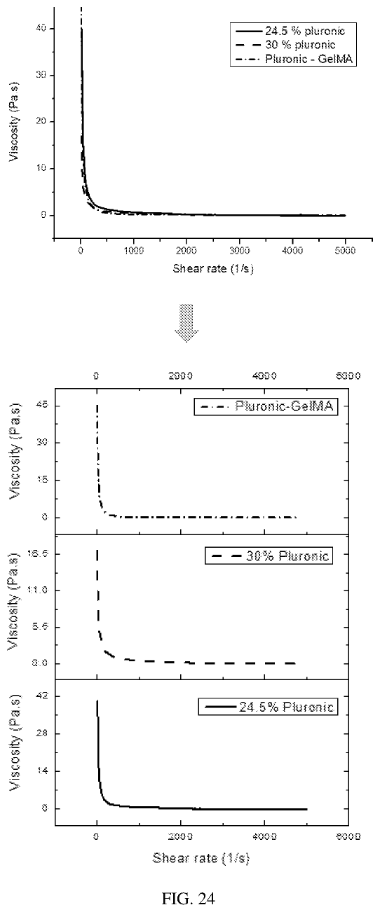

Rheological properties of Pluronic-GelMA were compared to 24.5% pluronic and 30% pluronic at room temperature from 0-5,000 1/s shear rate. The results in FIG. 24 showed that the pluronic-GelMA composite still retains thixotropic properties similar to pluronic, which is why the hydrogel provided good printability.

As shown in Table 2, the Plu-GelMA composite shows similar mechanical properties to human arteries in term of tensile strain.

TABLE 2
Mechanical properties of Plu-GelMA composite
compared to human arteries

| | Plu-GelMA | Human arteries |
|---|---|---|
| Compressive modulus (kPa) | 50.64 | N.A. |
| Compressive strain (mm) | 5.07 | N.A. |
| Compressive stress at Break | 7.86 | N.A. |
| (kPa) | | |
| Tensile strain (mm/mm) | 0.44 | 0.34 ± 0.10 |
| Tensile stress (kPa) | 18.31 | N.A. |

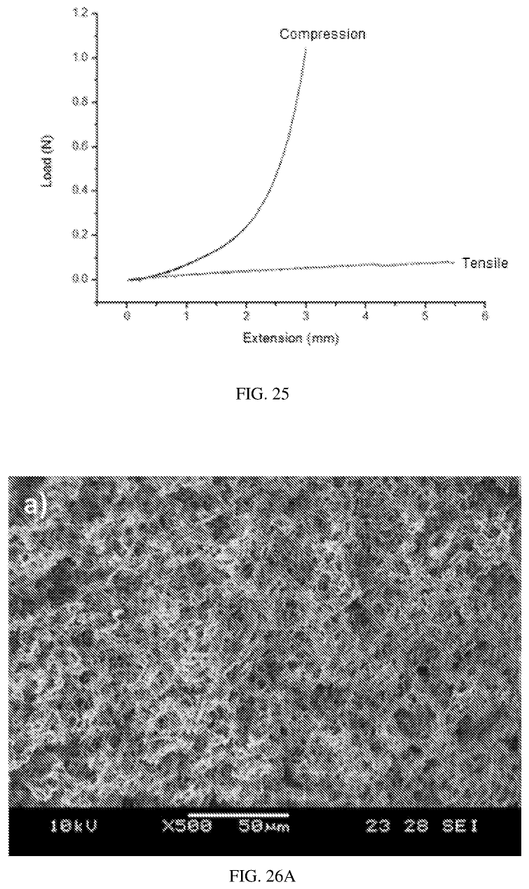

The noise of the mechanical curve which is shown in FIG. 25 came from porous properties which were shown in SEM images in the later section.

Mechanical properties of the hydrogel composite samples were tested at room temperature using a Model 5566 Instron universal testing machine (Instron, Norwood, Mass., USA). The hydrogel samples of each concentration were prepared for both compression and tensile test. The dog bone shaped samples (n=3, 10×15 mm) were prepared for tensile test while the circular dishes (n=3, Ø=10 mm) were prepared for compression test. Loads of 100 N and strain rate of 1 mm/min were applied. The results were shown in FIG. 9A to FIG. 9D.

Diffusion Test

The FITC-dextran (4 kDa, Sigma) solution at concentration levels of 0.0001, 0.0005, 0.001, 0.005, 0.01 and 0.05 mg/ml were analysed with microplate reader (SPARK 10M, Tecan) at excitation 485 nm, emission 535 nm) for generating standard curve. The results and curve fitting were shown in FIG. 10A.

In order to test the porosity level of each hydrogel composite concentration, the diffusion test used was similar to the FITC-dextran loading and releasing, which was reported by Martin et al. Briefly, dried composite hydrogels (≈40 mg, n>3) were dispersed in FITC-dextran (4 kDa, Sigma) solutions at a concentration of 2.0 mg/ml. After shaking for two hours for homogeneous absorption, the hydrogel samples were placed in PBS followed by shaking to ensure equal diffusion. The supernatant (PBS solution with FITC-dextran loaded sample) was taken out every hour from 0-4 hours to check the amount of FTIC-dextran diffused. The supernatant was later analysed with a microplate reader (SPARK 10M, Tecan) at excitation 485 nm and emission 535 nm. The results were shown in FIG. 10B.

Perfusion Properties

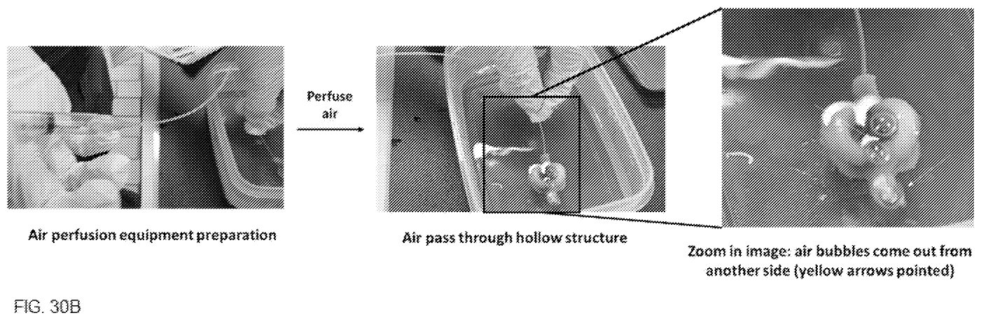

As shown in FIGS. 30A and 30B, coloured liquid and air were able to pass through and came out at the other end of bioprinted branches structure. This showed that this structure is truly hollow and can be used for perfusion applications.

Swelling Test

Hydrogel composite samples were casted into circular disc (n=3, Ø=10 mm, height=5 mm). After that, samples were dried by using freeze dryer and kept at −80° C. before undertaking further experiments. Subsequently, hydrogel samples were soaked in DI water for 4 hours to ensure that they were fully swollen and the swell ratio of hydrogel was calculated by using formula (II) below:

$$Swelling ratio = \frac{{W}_{swollen} - {W}_{dry}}{{W}_{dry}} \times 100 \% . \qquad Formula ( II )$$

Microstructure: Scanning Electron Microscope (SEM)

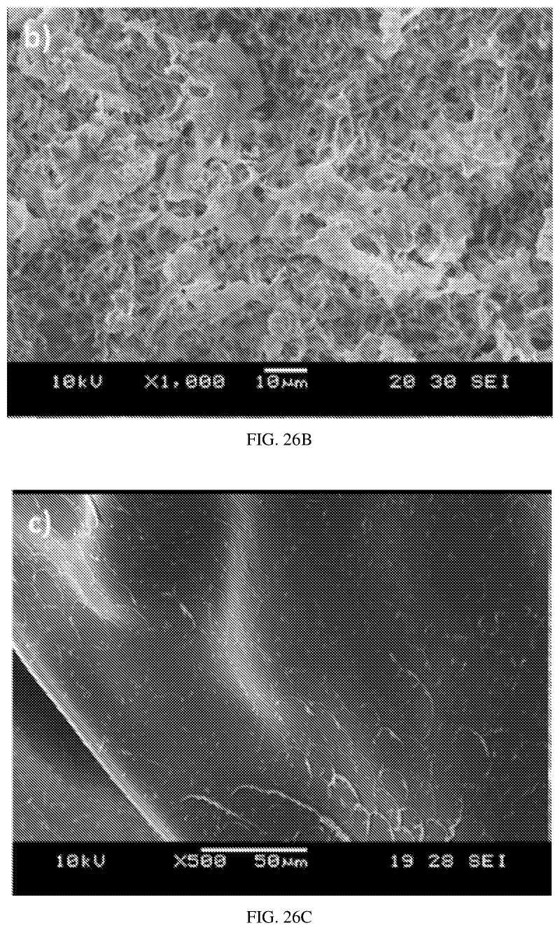

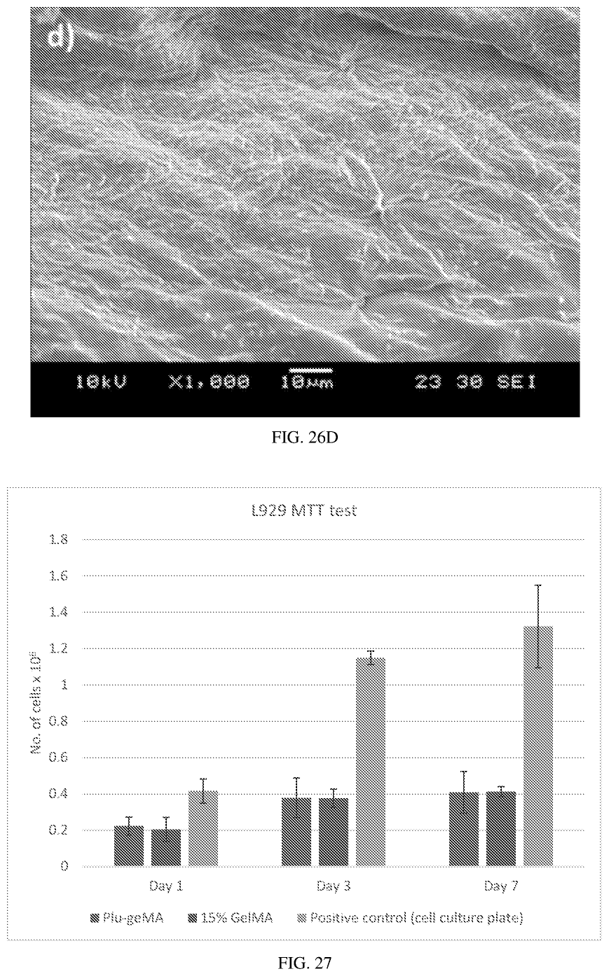

As shown in FIG. 26A to FIG. 26D, Plu-GelMA composite was extremely porous compared to GelMA. This type of microstructure can prove that this composite has “hydrogel sponge” texture. The sponge texture is similar to an extra-cellular matrix (ECM), which is favorable for tissue engineering application.

### EXAMPLE 6

### Enzymatic Degradation Test and Hydrogel Microstructure

As shown in FIG. 3, by using 0.002% w/v collagenase (2.5 U/ml), the enzymatic degradation of Plu-GelMA composite hydrogel showed that this composite is able to completely degrade within 10 days with a faster rate compared to 7.5% GelMA, but slower compared to 15% GelMA.

0.02 wt % Collagenase (Sigma-Aldrich, USA) in PBS was used for enzymatic degradation test of hydrogel composites over 16 days. Hydrogel samples were dried and the weight loss was checked every two days. Collagenase solution was changed every 2-3 days over the experimental period. As shown in FIG. 11A, all the composites fully degraded within 16 days. As expected, the greater GelMA content led to a slower degradation rate due to more chemically crosslinked bonds. Similar to most GelMA hydrogel bases, the mass of the hydrogel composite significantly dropped from day 1 to day 6 and the drop became slower afterwards. An error of this test is observed in some of the composites (1:1.5, 1:1 and 1.5:1), whereby the graphs for these composites show a mass increase from day 6 to day 8. This may be caused by the lack of protein coverage on the surface, which made the PEG component from Pluronic more dominant, hence leading to a change in swelling ratio. This might have caused the mass increase for a short while. The change in swelling ratio may be the reason of the increased mass at that point of time. However, after Pluronic dispersed loosely from the micelles packing, the degradation trend from day 8 onwards returned to normal. SEM images, after fixation as described later, were recorded and were shown in FIGS. 11B to 11D.

### EXAMPLE 7

### In Vitro Evaluation of Cell Viability and Cell Proliferation

In Vitro Evaluation of Hydrogel Composite

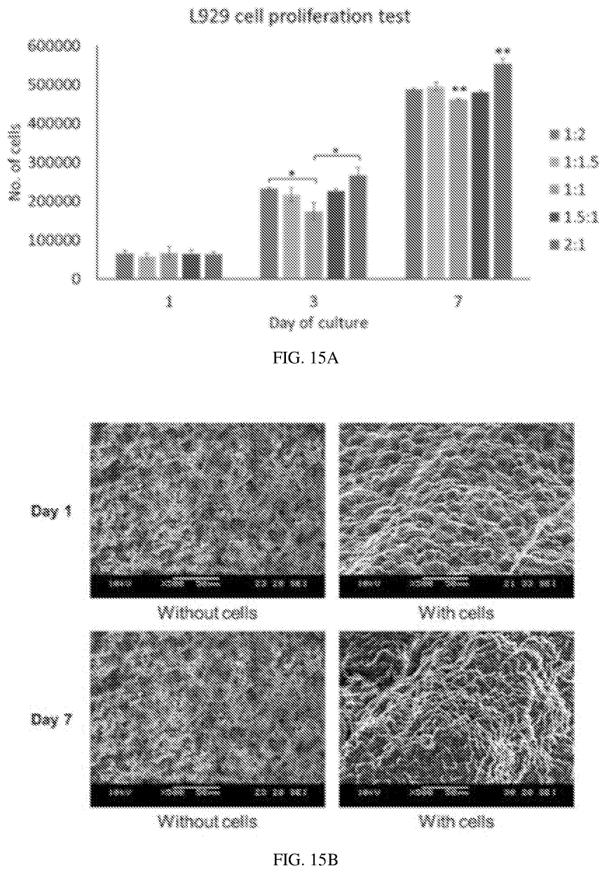

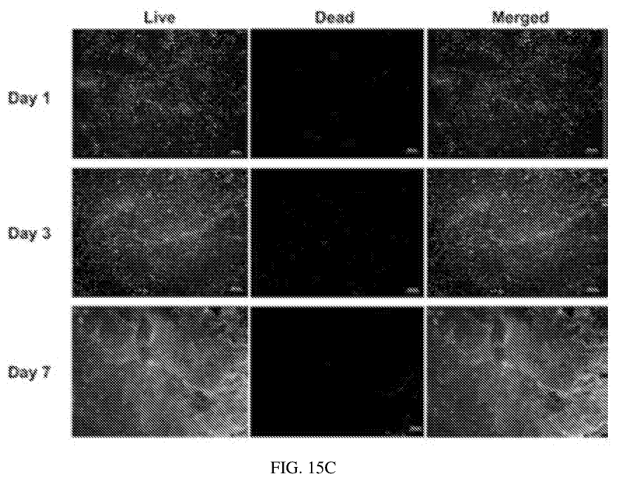

L929 fibroblast cells are commonly used for preliminary biocompatibility and toxicity tests. As shown in FIG. 15A, all Plu-GelMA of different mass ratios were biocompatible and supported cell proliferation, among which Plu-GelMA 2:1 achieved the highest number of cells at day 7. This can be further evidenced by SEM images (FIG. 15B) and live/dead staining (FIG. 15C) of L929 fibroblasts on Plu-GelMA 2:1.

Cell Compatibility and Immunofluorescent

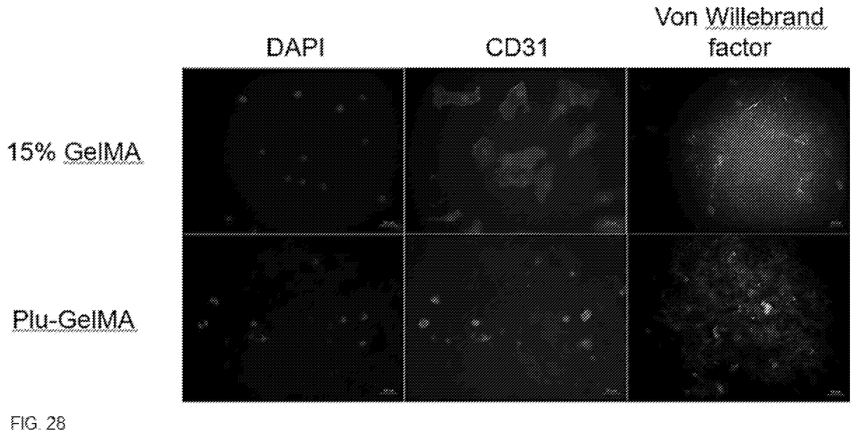

As mentioned above, L929 Fibroblasts are cell lines that are often used as first cell compatibility and toxicity test. In FIG. 27, it was shown that Plu-GelMA hydrogel composite provided similar compatibility as GelMA. The two hydrogels showed less absorbance signal, which may be due to absorption properties, wherein they absorb some of the MTT dye into themselves. Moreover, due to fully spreading, cells did not have enough space to continue proliferate on day 7.

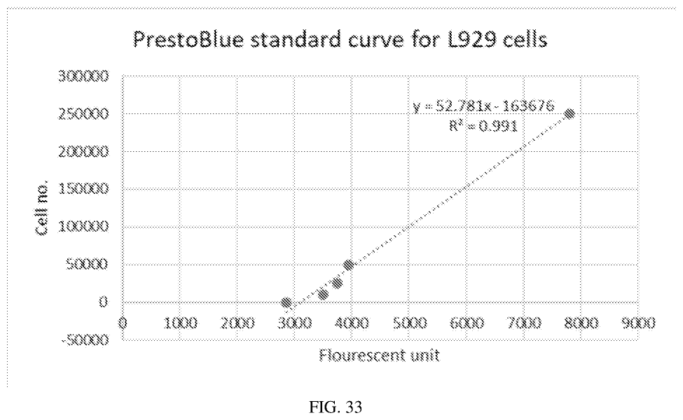

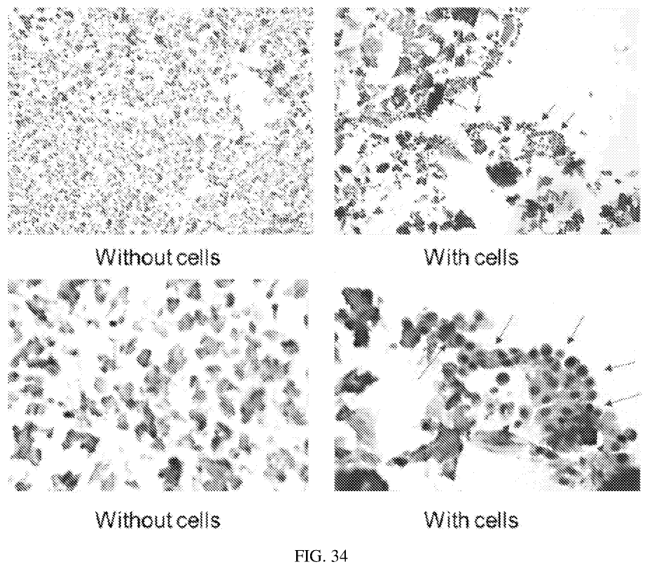

In total, 25 samples of 10 mm circular dish bioprinted hydrogels (five samples for each concentration) were sterilized by an autoclave at 80° C. for 15 minutes. Subsequently, samples were plated on a 24-well plate and soaked in cell culture medium for 12 hours before cell culture experiment. L929 mouse fibroblast cells were seeded at the density of 5×104 cells/well. Cells were cultivated in low glucose Dulbecco's Modified Eagle's Medium (DMEM) (Sigma-Aldrich) supplemented with 10%(v/v) FBS (PAA, GE Healthcare) and 1%(v/v) antibiotic/antimycotic solution (PAA, GE Healthcare). Culture medium was replaced after every 2-3 days and cells were grown at 37° C. in the presence of 5% CO₂. The experiment was stopped at day 1, 3 and 7 for live-dead staining, PrestoBlue® test and SEM fixation. For live-dead staining, the cell culture medium was removed from the sample, followed by washing the samples with PBS for 2-3 times. Live/Dead Cell Double Staining Kit (Sigma-Aldrich, USA) was used to validate cell compatibility of the hydrogels. Solution A (Calcein AM solution) and solution B (propidium iodide solution) were added to each sample with the ratio of 2:1 in PBS solution to form an assay solution. 100 μl of assay solution was added into each well and the well was incubated at 37° C. for 15 minutes. Then, cell viability was detected under fluorescence light using a fluorescence microscope with 490 nm excitation for live cells and at 545 nm excitation, whereby only dead cells can be observed. For PrestoBlue® test, the cell culture experiment was stopped by adding PrestoBlue® solution (Invitrogen, Life Technologies, USA) into cell culture 346 medium using a ratio of 1:9 by volume and the well was incubated at 37° C. for 2 hours. Then, the cell number was detected by using a microplate reader (SPARK 10M, Tecan) with 560 nm excitation and at 590 nm emission. The standard test compared to cell number is shown in the FIG. 33. For SEM fixation, the cell culture medium was removed to stop the experiment and the samples were washed with PBS 2-3 times. To investigate the cell morphology on the hydrogel composite, the samples needed to be viewed under scanning electron microscope (SEM). Before being able to do that, the samples needed to undergo SEM fixation as described below.

SEM Fixation

For primary fixation, 2.5% (v/v) glutaraldehyde solution (Sigma-Aldrich, USA) was used. All samples were soaked in 2.5% glutaraldehyde solution for 1 hour at 4° C. After that, samples were washed with distilled water for several times to remove glutaraldehyde. Next, ethanol was used to dehydrate the cells at a series of concentrations (v/v): 25%, 50%, 70%, 95%, 100% and 100%. Samples were soaked in each ethanol concentration for 10 minutes. After that, samples were washed with distilled water and dried in a desiccator for 1 day. Next, the hydrogel samples were coated with gold at 10 mA for 20 seconds before the SEM examination.

H&E of L929 Seeded Samples and the Cross-Section of Hybrid Structure

The H&E test is to stain nuclei of the cells and the protein for fixed tissue or ex vivo tissue samples. However, nowadays, it has been used for investigating cell-material interaction and dual-material interaction. As shown in FIG. 33, for the L929 seeded samples on 2:1 Plu-GelMA hydrogel composite, cells were able to attach and stay in the hydrogel, as the nucleus of L929 cells can be clearly seen in the sample.

### EXAMPLE 8

### In Vitro Evaluation for Cell Differentiation and Immunostaining

HUVECs are commonly used for angiogenesis study and vascular tissue engineering. In this research, they were used to further evaluate Plu-GelMA for supporting cell differentiation. Actin and collagen type IV immunofluorescence as well as SEM images (FIGS. 16A to 16C) showed that HUVECs were able to attach and spread on the Plu-GelMA 2:1 surface, and at day 7, they fused and formed layers covering the hydrogel surface. After 10 days of culture, the live/dead staining and immunofluorescence results (FIG. 16A) showed that HUVECs were fused and alive until day 10 and endothelium cell markers CD31 and VWF were expressed.

As shown in FIG. 28, both samples showed a clear signal of HUVEC nucleus (DAPI). CD31 and VWF are endothelium cells marker. The immunofluorescent staining of CD31 and VWF showed that endothelial cells were able to grow on both hydrogels efficiently. However, due to sponge properties and opacity, it is more difficult to observe this staining on Plu-GelMA compared to GelMA.

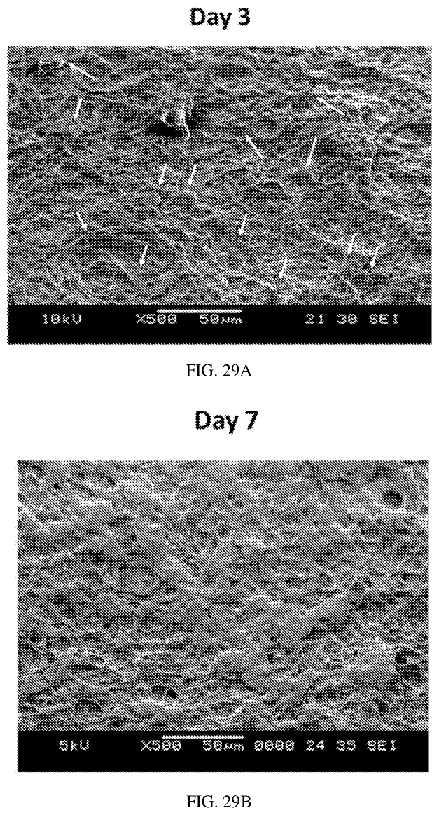

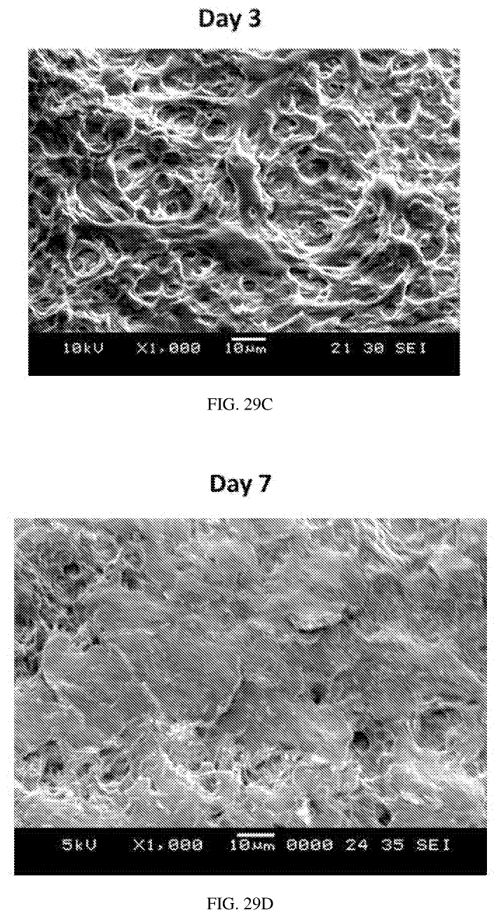

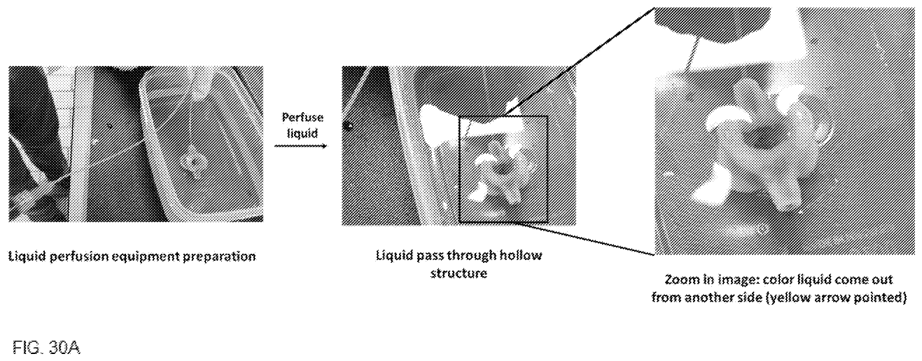

As shown in FIG. 29A to FIG. 29D, at day 3, cells were proliferated and spread onto the pluronic-GelMA surface. However, on day 7, cells were fused and combined. This result shows that this composite is not toxic to cells and also promotes cell proliferation. At day 7, the cells may not be able to further proliferate due to the limited surface area, and started to combine.

In total, nine samples of 10 mm circular dish bioprinted hydrogels (three samples for each concentration) were sterilized by utilizing an autoclave at 80° C. for 15 minutes. Subsequently, samples were plated on a 24-well plate and soaked in cell culture medium for 12 hours before cell culture experiment. HUVECs (Human Umbilical Vein Endothelium primary cells, Lonza) passage 5 were seeded at a density of 105 cells/well. Cells were cultivated in endothelial growth BulletKit (EGM-2, Lonza) and supplemented with 1% antibiotic/antimycotic solution (PAA, GE Healthcare). Culture medium was replaced after every 2-3 days and cells were grown at 37° C. in the presence of 5% CO₂. The live-dead staining protocol is the same as the protocol used for the L929 fibroblast cells. For the first two types of immunostaining, which were actin and collagen type IV, the HUVECs cells were cultured up to day 7. On the other hand, the cell differentiation of HUVECs was investigated at day 10 by using CD31 and von Willebrand Factor (VWF) expression. The samples were rinsed in DPBS a few times and fixed in 4% formaldehyde solution (Sigma-Aldrich, USA) in Dulbecco's Phosphate Buffered Saline (DPBS) (Hyclone, GE life science) for 30 minutes. After that the samples were soaked in blocking solution (5 wt % BSA, 0.5 wt % Tween in DPBS) for 2 hours at room temperature. Subsequently, the cell membranes were permeabilized in 0.25% (v/v) Triton X-100 (Bio-Rad, USA) in blocking solution for 20 minutes and washed with DPBS for three times. The samples were soaked in the primary antibody staining with 1/150 dilution of Rabbit polyclonal to Collagen IV (ab6586, abcam), 1/100 dilution of mouse monoclonal anti-CD31 antibody (Life technologies, Thermo fisher) and 3 μg/ml of VWF mouse monoclonal antibody (Life technologies, Thermo fisher) in DPBS overnight at 4° C. The samples were washed with blocking solution three times with 5 minutes intervals in between the washing steps. After primary antibody staining, the samples were incubated in 1/500 dilution of Alexa Fluor® 568 conjugated goat antirabbit (ab175471, abcam), 1/1000 dilution of Alexa Fluor-488 conjugated goat antimouse (Life technologies, Thermo fisher) and 1/500 dilution of Alexa Fluor-555 conjugated goat antimouse secondary antibodies (Life technologies, Thermo fisher) in DPBS for 2.5 hours at ambient condition (Alexa Fluor® 568 was paired with collagen IV, Alexa Flour 488 was paired with VWF and Alexa Flour 555 was paired with CD-31). Subsequently, the samples were washed in blocking solution three times with 15 minutes intervals in between the washing steps, followed by 5 μl/ml of Actin (ActinGreen™ ReadyProbes™, Thermo Fisher) and 5 μl/ml of DAPI (NucBlue®, Thermo Fisher) staining for 20 min. After rinsing, fluorescent images were taken by using a fluorescent microscope (Axio Vert.A1, Carl Zeiss, Germany). For SEM fixation of HUVECs, the samples were stopped at day 1 and day 7 and the protocol as mentioned above was followed.

PrestoBlue Standard Curve

PrestoBlue was used for testing different numbers of cells, from 10,000-250,000 cells, of L929 fibroblasts, to provide the standard curve shown in FIG. 33.

Commercial Applications of the Disclosure

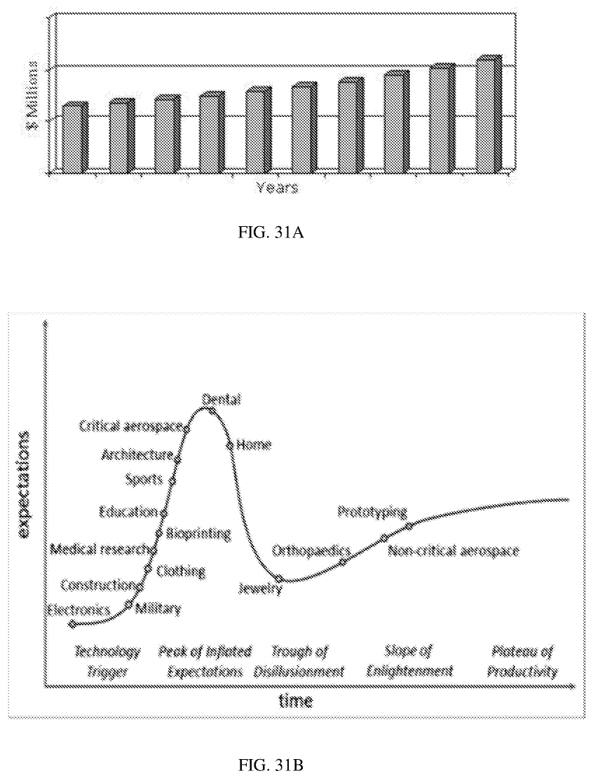

Bioprinting and biomaterials are emerging technologies that show tremendous economic potential. The market of biomaterials has been increasing every year. Biomaterials Market is expected to be worth $88.4 billion globally in 2017. On the other hand, the market for 3D bio-printing is to worth more than $3 billion as shown in FIG. 31A.

The present disclosure has various features which are high performance in printability for complex structure, high mechanical strength for in vitro and tissue model applications and easy to clean only by changing temperature (before crosslinking). Moreover, the product can be scaled up for large volume production.

The bio-ink offers superior performance which makes it suitable for complex 3D bioprinting. The 3D bioprinted part is very useful for tissue engineering and related biomedical applications and research, such as toxicity testing and drug delivery. Lastly, the high performance hydrogel may allow printing or guiding the blood vessels network structure, which may provide a possible alternative solution for vascularization tissue. The technique that uses two printable hydrogels allows the 3D printing of overhanging shapes to be fabricated on the solid platform, which is very unique and provides for advancement in bioprinting and biomedical fields.

The examples will be described in more detail below, along with supporting results.

### EXAMPLE 9

### Statistical Analysis

The statistical significance was determined by a Student t-test study for two groups of data or analysis of variance. P-values were presented as statistically significant and highly significant at 95% level of confidence as *P<0.05. ** P<0.05 is for data significantly different from the rest.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A hydrogel composite comprising a modified poloxamer having a first charged moiety and a peptide having a second charged moiety, wherein the first charged moiety and the second moiety are oppositely charged for ionic interaction between the modified poloxamer and the peptide, and wherein at least one of the modified poloxamer and the peptide comprises a crosslinkable moiety.

2.-3. (canceled)

4. The hydrogel composite according to claim 1, wherein the modified poloxamer has the general formula (I) 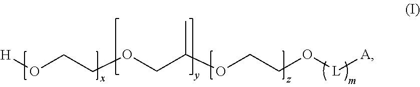
wherein x, y and z are independently integers in the range of about 2 to about 300, m is 0 or 1, L is a linker and A is the first charged moiety.

5.-8. (canceled)

9. The hydrogel composite according to claim 4, wherein A is selected from the group consisting of a carboxylate (—COO⁻), a deprotonated hydroxamic acid (—C(O)NHO⁻), a phosphate (—OPO₃²⁻), a sulfate (—OSO₃⁻) and any combination thereof.

10. (canceled)

11. The hydrogel composite according to claim 4, wherein L is selected from the group consisting of an optionally substituted C₁₋₂₀alkyl, optionally substituted C₂₋₂₀alkylene, optionally substituted C₆₋₁₂aryl, which is optionally substituted or interrupted with an amine, ether, ester, carbonyl, and any combination thereof.

12. (canceled)

13. The hydrogel composite according to claim 1, wherein the modified poloxamer has a number average molecular weight in the range of about 5 to about 20 kDa, preferably about 12.5 kDa.

14. The hydrogel composite according to claim 1, wherein the peptide is selected from the group consisting of sericine, fibroin, elastin, collagen, gelatin and a combination thereof.

15. The hydrogel composite according to claim 1, wherein the peptide is gelatin.

16. (canceled)

17. The hydrogel composite according to claim 1, wherein the peptide comprises an amino acid having a side chain, and the second charged moiety of the peptide is located on the side chain of the amino acid.

18.-21. (canceled)

22. The hydrogel composite according to claim 1, wherein the crosslinkable moiety is a methacrylate moiety.

23.-26. (canceled)

27. The hydrogel composite according to claim 1, wherein the mass ratio of the modified poloxamer to the peptide is in the range of about 0.5:5 to about 5:0.5, preferably about 1:1 to about 3:1, more preferably at about 2:1.

28. A process for making a hydrogel composite, the process comprising:
a) providing a modified poloxamer having a first charged moiety and a peptide having a second charged moiety, wherein the first charged moiety and the second charged moiety are oppositely charged for ionic interaction between the modified poloxamer and the peptide, and wherein at least one of the modified poloxamer and the peptide comprises a crosslinkable; and
b) mixing the modified poloxamer with the peptide and allowing the modified poloxamer and the peptide to ionically interact to obtain the hydrogel composite.

29.-33. (canceled)

34. The process according to claim 28, wherein the peptide is provided in the form of uncoiled chains of a peptide backbone.

35. The process according to claim 28, wherein the mixing is carried out using a three-way stopcock.

36. A kit of parts for making a hydrogel composite according to claim 1, comprising a first part of a modified poloxamer having a first charged moiety and a second part of a peptide having a second charged moiety, wherein the first charged moiety and the second charged moiety are oppositely charged for ionic interaction between the modified poloxamer and the peptide, and wherein at least one of the modified poloxamer and the peptide comprises a crosslinkable moiety.

37. A bioshaping method comprising providing the hydrogel composite according to claim 1, shaping the hydrogel composite to obtain a shaped hydrogel composite, and carrying out a cross-linking reaction on the shaped hydrogel composite to obtain a three-dimensional crosslinked network.

38.-44. (canceled)

45. A three-dimensional crosslinked network obtainable using the bioshaping method according to claim 37.

46. A method of treating vascularization insufficiency comprising implanting a three-dimensional crosslinked network according to claim 45 into a mammal's body.

47. The hydrogel composite according to claim 1, wherein a peptide backbone of the peptide is in the form of a linear chain.

48. The process according to claim 28, wherein a peptide backbone of the peptide is provided in the form of a linear chain.

49. The kit of parts according to claim 36, wherein a peptide backbone of the peptide is in the form of a linear chain.
